(19) United States
(12) Patent Application Publication
(10) Pub. No.: US 2026/0051362 A1
(43) Pub. Date: Feb. 19, 2026

(54) GENERATIVE PROTEIN DESIGN WITH SMOOTHED ENERGY-BASED MODELS

(71) Applicant: Genentech, Inc., South San Francisco, CA (US)

(72) Inventors: Sai Pooja MAHAJAN, San Francisco, CA (US); Saeed SAREMI, South San Francisco, CA (US); Daniel BERENBERG, New York, NY (US); Richard A. Bonneau, New York, NY (US); Kyunghyun CHO, New York, NY (US); Nathan Christopher FREY, New York, NY (US); Vladimir GLIGORIJEVIC, New Brunswick, NJ (US)

(21) Appl. No.: 19/288,834

(22) Filed: Aug. 1, 2025

Related U.S. Application Data
(63) Continuation of application No. PCT/US2024/013935, filed on Feb. 1, 2024.
(60) Provisional application No. 63/482,756, filed on Feb. 1, 2023.
(60) Provisional application No. 63/502,497, filed on May 16, 2023.
(60) Provisional application No. 63/588,437, filed on Oct. 6, 2023.

(51) Int. Cl. G16B 15/20; G16B 30/00; G16B 40/20

(52) U.S. Cl. CPC G16B 15/20; G16B 30/00; G16B 40/20

(57) ABSTRACT

A training set may be generated to include a plurality of noisy sample sequences. Each noisy sample sequence in the training set may be generated by adding noise to a corresponding sample sequence from a data distribution. A protein design computation model may be trained by at least applying the protein design computation model to generate one or more output sequences, and adjusting the protein design computation model to reduce a difference between the one or more output sequences and the plurality of noisy sample sequences in the first training set. The trained protein design computation model may be applied to generate an output sequence by at least modifying an input sequence.

### CROSS REFERENCE TO RELATED APPLICATIONS

This application claims priority to U.S. Provisional Application No. 63/482,756, entitled “GENERATIVE PROTEIN DESIGN WITH SMOOTHED ENERGY-BASED MODELS” and filed on Feb. 1, 2023, U.S. Provisional Application No. 63/502,497, entitled “GENERATIVE PROTEIN DESIGN WITH SMOOTHED ENERGY-BASED MODELS” and filed on May 16, 2023, and U.S. Provisional Application No. 63/588,437, entitled “GENERATIVE PROTEIN DESIGN WITH SMOOTHED ENERGY-BASED MODELS” and filed on Oct. 6, 2023, the disclosures of which are incorporated herein by reference in their entireties.

### TECHNICAL FIELD

The subject matter described herein relates generally to computational protein design and more specifically to energy-based models (EBM) for generating protein sequences.

### INTRODUCTION

Proteins are genetically encoded macromolecules with tremendous diversity in size and chemical composition. By regulating biological systems, proteins facilitate many essential cellular functions including, for example, enzymatic reactions, molecular transport, regulation and execution of a number of biological pathways, cell growth, proliferation, nutrient uptake, morphology, motility, intercellular communication, and/or the like. A protein structure may include one or more polypeptides, each of which including a sequence of amino acid residues linked together by peptide bonds (e.g., covalent peptide bonds). There are twenty canonical amino acid residues which, unlike non-canonical amino acid residues, are encoded directly by the genetic code. Each canonical amino acid residue includes the same backbone atoms (e.g., an amino group (NH₂), an alpha carbon (C_{α}), and a carboxylic group (COOH)) coupled with a different combination of sidechain atoms (or R groups).

The primary structure of a protein molecule refers to the sequence of amino acid residues in each of the polypeptide chains forming the protein structure. The backbone atoms in adjacent amino acid residues that participate in the peptide bonds (e.g., covalent peptide bonds) therebetween form a repeating sequence of atoms known as the polypeptide backbone (or backbone) of the protein molecule. The secondary structure of the protein molecule refers to the local folded structures (e.g., α helixes, β pleated sheet, and/or the like) that form within an individual polypeptide chain due to interactions between the backbone atoms (e.g., amino hydrogen atoms, carboxyl oxygen atoms, and/or the like). Further interactions (e.g., non-covalent bonds such as hydrogen bonding, ionic bonding, dipole-dipole interactions, and van der Waals forces) between the sidechains (or R-groups) of the amino acid residues in the protein molecule may cause folding within the individual polypeptide chains, thus forming the tertiary structure of the protein molecule. The tertiary structure of the protein molecule is also known as the conformation or the three-dimensional structure of the protein molecule. In protein molecules having multiple polypeptide chains, the protein molecule may also exhibit a quaternary structure, which is formed when the polypeptide chains are packed and held together by hydrogen bonds and van der Waals forces (e.g., between nonpolar sidechains).

The functions of a protein molecule may be contingent upon the sequence of amino acid residues in the polypeptide chains forming the protein molecule as well as the three-dimensional structure adopted by the polypeptide chains. For example, the primary structure of the protein molecule may determine the three-dimensional structure assumed by the protein molecule through the folding of the constituent polypeptide chains. In some cases, the binding affinity of the protein molecule towards a target molecule, such as a viral or tumor antigen, may depend on whether the polypeptide chains in the protein molecule are able to assume a three-dimensional structure that complements the three-dimensional structure of the target molecule and is sufficiently stable to allow a binding interaction between the two molecules. As such, one notable objective of computational protein design is to construct one or more protein sequences (e.g., antibodies and/or the like) that exhibit certain desirable properties. For instance, in the case of large molecule drug discovery (LMDD), computational protein design may seek to identify therapeutically viable protein sequences (e.g., antibodies and/or the like) with a variety of desirable properties such as expression, binding affinity towards a target molecule, binding specificity, stability, non-immunogenicity, human-ness, absence of self-association (or non-aggregation), lack of chemical liabilities (e.g., aspartate isomerization, oxidation, deamidation), and/or the like.

### SUMMARY

Systems, methods, and articles of manufacture, including computer program products, are provided for generative protein design in which an energy-based model (EBM) is applied to generate protein sequences. In one aspect, there is provided a system for generative protein design that includes at least one processor and at least one memory. The at least one memory may include program code that provides operations when executed by the at least one processor. The operations may include: generating a first training set to include a plurality of noisy sample sequences, each noisy sample sequence in the first training set being generated by at least adding noise to a corresponding sample sequence from a first data distribution; training a protein design computation model by at least applying the protein design computation model to generate one or more output sequences, and adjusting the protein design computation model to reduce a difference between the one or more generated output sequences and the plurality of noisy sample sequences in the first training set; applying the trained protein design computation model to generate an output sequence by at least modifying an input sequence.

In another aspect, there is provided a method for generative protein design. The method may include: generating a first training set to include a plurality of noisy sample sequences, each noisy sample sequence in the first training set being generated by at least adding noise to a corresponding sample sequence from a first data distribution; training a protein design computation model by at least applying the protein design computation model to generate one or more output sequences, and adjusting the protein design computation model to reduce a difference between the one or more generated output sequences and the plurality of noisy sample sequences in the first training set; applying the trained protein design computation model to generate an output sequence by at least modifying an input sequence.

In another aspect, there is provided a computer program product including a non-transitory computer readable medium storing instructions. The instructions may cause operations may executed by at least one data processor. The operations may include: generating a first training set to include a plurality of noisy sample sequences, each noisy sample sequence in the first training set being generated by at least adding noise to a corresponding sample sequence from a first data distribution; training a protein design computation model by at least applying the protein design computation model to generate one or more output sequences, and adjusting the protein design computation model to reduce a difference between the one or more generated output sequences and the plurality of noisy sample sequences in the first training set; applying the trained protein design computation model to generate an output sequence by at least modifying an input sequence.

In some variations, one or more features disclosed herein including the following features can optionally be included in any feasible combination.

In some variations, the protein design computation model includes a first energy-based model (EBM).

In some variations, the training of the protein design computation model includes adjusting a plurality of parameters of the first energy-based model parameterizing an energy function of the first energy-based model.

In some variations, the plurality of parameters are adjusted such that an energy value determined by the energy function corresponds to a likelihood of the one or more generated output sequences within the first data distribution.

In some variations, the plurality of parameters are adjusted such that the energy function outputs a lower energy value for a first generated output sequence that is more similar to the plurality of noisy samples in the first training set than a second generated output sequence that is less similar to the plurality of noisy samples in the first training set.

In some variations, the training of the protein design computation model includes applying the first energy-based model having a first adjustment to generate a first modified sequence, applying the first energy-based model having a second adjustment to generate a second modified sequence, and upon determining that the first modified sequence is more similar to the plurality of noisy samples in the first training set than the second modified sequence, further modifying the first energy-based model having the first adjustment instead of the second adjustment.

In some variations, the first energy-based model is further adjusted until one or more criteria are met. The one or more criteria include at least one of (i) having performed a threshold quantity of iterations of adjustments to the first energy-based model and (ii) the second modified sequence exhibiting a threshold similarity to the plurality of noisy samples in the first training set.

In some variations, the protein design computation model further includes a second energy-based model (EBM).

In some variations, a second training set including a plurality of sample sequences from a second data distribution may be generated. A first adjustment to the first energy-based model that reduces a first difference between a first output sequence generated by the first energy-based model and the plurality of noisy sample sequences in the first training set may be determined. A second adjustment to the second energy-based model that reduces a second difference between a second output sequence generated by the second energy-based model and the plurality of sample sequences in the second data distribution may be determined. The first energy-based model may be trained by at least applying, to the first energy-based model, a third adjustment determined based on the first adjustment and the second adjustment.

In some variations, the third adjustment corresponds to a sum or a weighted sum of the first adjustment and the second adjustment.

In some variations, each sample sequence from the first data distribution may be encoded to generate an embedding of each sample sequence. The plurality of noisy sample sequences in the first training set may be generated by at least adding noise to the embedding of each sample sequence.

In some variations, each sample sequence from the first data distribution is encoded by being enriched with additional information.

In some variations, the additional information includes structural information that identifies, for each constituent amino acid residue, one or more neighboring amino acid residue in three-dimensional space.

In some variations, the trained protein design computation model generates the output sequence by at least generating a noisy input sequence by at least adding noise to the input sequence, applying an energy-based model to generate a noisy output sequence by at least modifying, based at least on an energy function of the energy-based model, the noisy input sequence, and generating the output sequence by at least denoising the modified noisy output sequence generated by the energy-based model.

In some variations, the trained protein design computation model generates the output sequence by at least generating an embedding of the input sequence by at least encoding the input sequence, generating a noisy embedding of the input sequence by at least adding noise to the embedding of the input sequence, applying an energy-based model to generate a modified noisy embedding by at least modifying, based at least on an energy function of the energy-based model, the noisy embedding of the input sequence, denoising the noisy embedding to generate a denoised embedding, and generating the output sequence by at least denoising the noisy embedding.

In some variations, the embedding of the input sequence is generated by at least generating, for each amino acid residue in the input sequence, a token encoding an identity of the amino acid residue.

In some variations, the embedding of the input sequence is generated by at least generating one or more structural tokens identifying, for at least one amino acid residue in the input sequence, one or more neighboring amino acid residue in three-dimensional space.

In some variations, the trained protein design computation model modifies the input sequence by at least one of (i) inserting an amino acid residue, (ii) deleting an amino acid residue, and (iii) changing an identity of an amino acid residue in the input sequence.

In some variations, a fixed-length representation of the input sequence may be generated. The trained protein design computation model may be applied to generate the output sequence by at least modifying the fixed length representation of the input sequence.

In some variations, the fixed-length representation of the input sequence is generated by at least aligning each amino acid residue in the input sequence to a fixed set of structural roles such that each amino acid residue in the input sequence is assigned an integer position corresponding to a structural role of the amino acid residue, and inserting a gap character at one or more positions where the input sequence fails to include an amino acid residue having a corresponding structural role.

In some variations, the difference between the one or more generated output sequences and the plurality of noisy sample sequences is quantified by one or more of an antibody likeness metric, an edit distance, and a naturalness metric.

In another aspect, there is provided a system for generative protein design that includes at least one processor and at least one memory. The at least one memory may include program code that provides operations when executed by the at least one processor. The operations may include: identifying an input sequence having a plurality of amino acid residues; generating a noisy embedding of the input sequence by at least adding noise to the input sequence; modifying the noisy embedding of the input sequence by at least applying a protein design computation model trained to approximate a data distribution of protein sequences exhibiting one or more desirable properties, the protein design computation model modifying the noisy embedding of the input sequence to increase a likelihood of a modified noisy embedding resulting therefrom being in the data distribution; and generating an output sequence by at least denoising the modified noisy embedding generated by the protein design computation model.

In another aspect, there is provided a method for generative protein design. The method may include: identifying an input sequence having a plurality of amino acid residues; generating a noisy embedding of the input sequence by at least adding noise to the input sequence; modifying the noisy embedding of the input sequence by at least applying a protein design computation model trained to approximate a data distribution of protein sequences exhibiting one or more desirable properties, the protein design computation model modifying the noisy embedding of the input sequence to increase a likelihood of a modified noisy embedding resulting therefrom being in the data distribution; and generating an output sequence by at least denoising the modified noisy embedding generated by the protein design computation model.

In another aspect, there is provided a computer program product including a non-transitory computer readable medium storing instructions. The instructions may cause operations may executed by at least one data processor. The operations may include: identifying an input sequence having a plurality of amino acid residues; generating a noisy embedding of the input sequence by at least adding noise to the input sequence; modifying the noisy embedding of the input sequence by at least applying a protein design computation model trained to approximate a data distribution of protein sequences exhibiting one or more desirable properties, the protein design computation model modifying the noisy embedding of the input sequence to increase a likelihood of a modified noisy embedding resulting therefrom being in the data distribution; and generating an output sequence by at least denoising the modified noisy embedding generated by the protein design computation model.

In some variations, one or more features disclosed herein including the following features can optionally be included in any feasible combination.

In some variations, the input sequence is encoded to generate an embedding of the input sequence. The noisy embedding of the input sequence is generated by at least adding noise to the embedding of the input sequence. The output sequence is generated by decoding a denoised embedding generated by the denoising of the modified noisy embedding.

In some variations, the input sequence is encoded by at generating, for each amino acid residue in the input sequence, a token encoding an identity of the amino acid residue.

In some variations, the input sequence is encoded by at least generating one or more tokens encoding a relative position of each amino acid residue within the input sequence.

In some variations, the input sequence is encoded by at least generating one or more structural tokens identifying, for at least one amino acid residue in the input sequence, one or more neighboring amino acid residue in three-dimensional space.

In some variations, the modifying of the noisy embedding includes applying an energy-based model (EBM) trained to approximate the data distribution to modify the noisy embedding of the input sequence and generate a first modified noisy embedding, applying the energy-based model (EBM) to modify the noisy embedding of the input sequence and generate a second modified noisy embedding, applying an energy function parameterized by the energy-based model (EBM) to determine a first energy value of the first modified noisy embedding and a second energy value of the second modified noisy embedding, and applying the energy-based model (EBM) to further modify, based at least on the first energy value and the second energy value, the first modified noisy embedding instead of the second modified noisy embedding.

In some variations, the energy-based model (EBM) is applied to further modify the first modified noisy embedding until one or more criteria are met.

In some variations, the one or more criteria include at least one of (i) having performed a threshold quantity of iterations of modifications to the noisy embedding of the input sequence and (ii) the first energy value of the first modified noisy embedding satisfying one or more thresholds.

In some variations, the energy-based model (EBM) is applied to further modify the first modified nosy embedding instead of the second modified noisy embedding based at least on the first energy value and the second energy value indicating that the first modified noisy embedding has a higher likelihood of being in the data distribution than the second modified noisy embedding.

In some variations, the energy-based model (EBM) is applied to further modify the first modified nosy embedding instead of the second modified noisy embedding based at least on the first energy value and the second energy value indicating that the first modified noisy embedding is sampled from a higher density region of the data distribution than the second modified noisy embedding.

In some variations, a fixed-length representation of the input sequence is generated. The noisy embedding of the input sequence is generated based at least on the fixed-length representation of the input sequence.

In some variations, the fixed-length representation of the input sequence is generated by at least aligning each amino acid residue in the input sequence to a fixed set of structural roles such that each amino acid residue in the input sequence is assigned an integer position corresponding to a structural role of the amino acid residue, and inserting a gap character at one or more positions where the input sequence fails to include an amino acid residue having a corresponding structural role.

In some variations, the protein design computation model modifies the noisy embedding of the input sequence by at least one changing an identity of one or more amino acid residues in the input sequence, deleting an amino acid residue occupying a position within the fixed-length representation of the input sequence by at least replacing the amino acid residue with a gap character, and inserting an amino acid residue at a position within the fixed-length representation of the input sequence by at least replacing a gap residue occupying the position with the amino acid residue.

In some variations, the one or more desirable properties include at least one of expression, affinity, specificity, stability, non-immunogenicity, human-ness, absence of self-association, and lack of chemical liabilities.

In some variations, the input sequence is a known protein sequence or a noise sequence comprising a random sequence of amino acid residues.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the computational design of protein molecules including protein-based therapeutics such as antibodies, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a protein design system, in accordance with some example embodiments;

FIG. 2A depicts a flowchart illustrating an example of a process for computational protein design, in accordance with some example embodiments;

FIG. 2B depicts a flowchart illustrating another example of a process for computational protein design, in accordance with some example embodiments;

FIG. 3A depicts a flowchart illustrating an example of a process for training a protein design computation model, in accordance with some example embodiments;

FIG. 3B depicts a flowchart illustrating another example of a process for training a protein design computation model, in accordance with some example embodiments;

FIG. 4A depicts a schematic diagram illustrating an example of a sampling from a noisy data distribution, in accordance with some example embodiments;

FIG. 4B depicts schematic diagrams illustrating a comparison of density estimation for a clean data distribution without any noise perturbation and density estimation for a noisy data distribution, in accordance with some example embodiments;

FIG. 5A depicts a schematic diagram illustrating an example of sampling from a smoothed discrete space, in accordance with some example embodiments;

FIG. 5B depicts a block diagram illustrating an example of a discrete energy-based model (dEBM), in accordance with some example embodiments;

FIG. 6 depicts a schematic diagram illustrating an example of sampling from a smoothed latent space, in accordance with some example embodiments;

FIG. 7A depicts a graph illustrating validation loss over successive training steps (or gradient updates) for different noise levels in the training data, in accordance with some example embodiments;

FIG. 7B depicts a graph illustrating the similarity distribution of antibody heavy chains and antibody light chains generated by a protein design computation model, in accordance with some example embodiments;

FIG. 7C depicts a graph illustrating the naturalness distribution of antibody heavy chains and antibody light chains generated by a protein design computation model, in accordance with some example embodiments; and

FIG. 8 depicts a schematic diagram illustrating a distributional conformity score based evaluation of the in silico protein designs generated by a protein design computation model relative to a reference set of validation samples, in accordance with some example embodiments;

FIG. 9 depicts a block diagram illustrating an example of a computing system, in accordance with some example embodiments.

When practical, similar reference numbers denote similar structures, features, or elements.

### DETAILED DESCRIPTION

Computational protein design aims to generate protein sequences that exhibit a variety of desirable properties. In the context of large molecule drug discovery (LMDD), for example, whether a protein sequence exhibits certain drug-like properties may determine its viability as a protein-based therapeutic such as an antibody, enzyme, growth factor, hormone, interferon, interleukin, thrombolytic, and/or the like. As such, in some cases, a drug development pipeline may include assessing a candidate protein sequence for the presence of drug-like properties. For example, a candidate protein sequence that successfully passes in vitro validation can then undergo preclinical development and clinical trials, where the performance of the candidate protein sequence is tested in vivo. However, the significant expense of wet lab resources means that a limited number of candidate protein sequences can proceed to in vitro and in vivo assessment. As such, one key objective of computational protein design is to increase (or maximize) the likelihood that computationally generated candidate protein sequences exhibit the drug-like properties necessary for successful in vitro and in vivo testing. For instance, as a protein-based therapeutic, a protein sequence may be computationally engineered to ensure that the protein sequence exhibits sufficient expression, affinity and targetability, in vivo stability, pharmacokinetics, cell permeability, and non-immunogenicity.

Computational protein design is a challenging and resource intensive task at least because numerous possible variations in protein sequence and conformation (or three-dimensional structure) exist but only a small fraction of these variants will have any therapeutic value. For example, of the 20^{L} possible protein sequences formed by an L-quantity of amino acid residues selected from the twenty canonical amino acid residues, few will have the combination of drug-like properties (e.g., affinity, specificity, biological activity, and developability) required for a protein-based therapeutic. Thus, increasing (or maximizing) the likelihood that a computationally generated protein sequence submitted as a candidate for in vitro and/or in vivo assessment exhibits drug-like properties may require evaluating at least some of the 20^{L} possible protein sequences. However, evaluating an arbitrary subset of the 20^{L} possible protein sequences for the presence of drug-like properties may inadvertently overlook at least some with better drug-like properties. Contrastingly, even when performed in silico, a brute force evaluation of every possible protein sequence is too computationally expensive to be a feasible solution. As such, in some example embodiments, a protein design computation model may explore the vast combinatorial space of possible protein sequences in a principled manner in order to identify candidate protein sequences with a higher likelihood of exhibiting the requisite combination of drug-like properties.

In some example embodiments, the protein design computation model may generate one or more protein sequences by at least sampling a data distribution populated by known protein sequences (e.g., from the Protein Data Bank (PDB)) or a certain subset thereof (e.g., the Observed Antibody Space (OAS)). In some cases, the known protein sequences (or the subset thereof) may exhibit one or more desirable properties including, for example, drug-like properties such as expression, affinity, specificity, stability, non-immunogenicity, human-ness, absence of self-association, lack of chemical liabilities, and/or the like. Furthermore, in some cases, high density regions of the data distribution may be populated by protein sequences similar to the known protein sequences exhibiting the one or more desirable properties while low density regions of the data distribution may be populated by protein sequences dissimilar to the known protein sequences. Accordingly, in some cases, the one or more protein sequences may be generated by sampling from the higher density regions of the data distribution, which are more likely to be populated by the protein sequences similar to the known protein sequences. To do so, the protein design computation model may undergo training to determine an energy function that approximates the data distribution. For instance, in some cases, the data distribution, in particular the gradient of the energy function approximating the different densities across the data distribution, may be determined through Bayesian inference. The protein design computation model may then sample the data distribution based on the gradient of the energy function such that the protein sequences are sampled from the higher density regions of the data distribution instead of the lower density regions of the data distribution, thus increasing the likelihood that the protein sequences exhibit the one or more desirable properties.

Training the protein design computation model to approximate the data distribution and sampling efficiently therefrom to generate novel, unique, diverse, and therapeutically viable protein sequences pose a number of unique challenges. At the outset, the data distribution of protein sequences is high-dimensional (e.g., 20^{L} dimensions for length L protein sequences) but disproportionately few known protein sequences characterizing the data distribution are available to train the protein design computation model. While the known protein sequences may identify some regions of high density within the data distribution, the densities of regions therebetween remain unknown. Consequently, the protein design computation model may be prone to overfitting where the protein design computation model is unable to generate protein sequences that are sufficiently diverse from the known protein sequences. In this case, the phenomenon of overfitting may arise due to the protein design computation model learning, based on the known protein sequences that are available, an energy function that fails to accurately capture the different densities of the data distribution between known protein sequences. For example, in some cases, the energy function may approximate a jagged energy landscape at least because the gradient of the energy function exhibits sharp changes corresponding to the stark differences in density that exist between the regions populated by known protein sequences where the density of the data distribution is indeterminate. That the energy function approximates a jagged energy landscape may prevent a sufficient exploration of the data distribution when the protein design computation model is subsequently applied to sample from the data distribution based on the gradient of the energy function. For instance, the protein sequences generated by the sampling of the data distribution may be repetitive and limited in variety at least because the gradient of the energy function restricts the protein design computation model to sample from within the immediate vicinity around known protein sequences.

In some example embodiments, the energy function approximating the densities across the data distribution of known protein sequences may be determined based on a noisy training set of sample sequences, each of which being a known protein sequence that has been adulterated with noise (e.g., isotropic Gaussian noise and/or the like). That is, instead of the training the protein design computation model to approximate the data distribution of the known protein sequences based on the known protein sequences directly, the protein design computation model may be trained to approximate the data distribution of the known protein sequences based on the noisy training set. Doing so may reduce the jaggedness of the energy landscape approximated by the energy function such that the protein design computation model is able to sample efficiently across the data distribution of known protein sequences. For example, in some cases, the protein design computation model may include at least one energy-based model (EBM). Training the protein design computation model to approximate the data distribution of known protein sequences determining, based on the noisy training set, an energy function that approximates the different densities across the data distribution. It should be appreciated that the energy function may be parametrized by the parameters of the energy-based model. For instance, in cases where the energy-based model (EBM) is implemented with an artificial neural network (e.g., a convolutional neural network and/or the like), the parameters of the energy function may correspond to the weights and/or biases applied by the neurons in each successive layer of the artificial neural network. Training the protein design computation model to learn the data distribution may include adjusting the parameters of the energy-based model (EBM) to increase the similarity between the protein sequences generated by the energy-based model (EBM) and the sample sequences in the noisy training set. In doing so, the parameters of the energy function may also be adjusted such that the energy function outputs, for each protein sequence, an energy indicative of whether the protein sequence is in or out of the data distribution.

In some example embodiments, the training of the protein design computation model may include gradient based Markov Chain Monte Carlo (MCMC) sampling (e.g., Markov Chain Monte Carlo sampling with Langevin dynamics and/or the like) in which the parameters of the energy-based model (EBM) and those of the corresponding energy function are adjusted over successive sampling iterations to increase the similarity between the protein sequences generated by the energy-based model (EBM) sampling from the data distribution and the sample sequences in the noisy training set. For example, gradient based Markov Chain Monte Carlo may include applying the energy-based model (EBM) to modify an input sequence, which may be a known protein sequence or a noise sequence (e.g., a sequence of random amino acid residues), to generate a first modified sequence before applying the energy-based model (EBM) to further modify the first modified sequence to generate a second modified sequence. In some cases, the energy-based model (EBM) may be applied again to further modify the second modified sequence and generate a third modified sequence. The parameters of the energy-based model (EBM) may be adjusted to such that the second modified sequence is more similar to the sample sequences in the noisy training set than the first modified sequence. In some cases, the parameters of the energy-based model (EBM) may be further modified such that the third modified sequence is more similar to the sample sequences in the noisy training set than the second modified sequence. As noted, adjusting the parameters of the energy-based model (EBM) also adjusts those of the corresponding energy function. For instance, in some cases, the parameters of the energy function may undergo successive adjustments to lower the energy value output by the energy function for protein sequences that are within the data distribution of the known protein sequences.

In some example embodiments, to avoid overfitting the protein design computation model to the known protein sequences, the protein design computation model may be trained based on a noisy training set of known protein sequences that have been adulterated with noise and not the known protein sequences directly. For example, in some cases, the training of the protein design computation model may include a gradient based Markov Chain Monte Carlo (MCMC) sampling (e.g., Langevin Markov Chain Monte Carlo (MCMC) sampling and/or the like) in which the parameters of the energy-based model (EBM) are adjusted over successive sampling iterations increase the similarity between the protein sequences sampled from the data distribution and the sample sequences in the noisy training set. The energy function derived in this manner based on the noisy training set may be parameterized to capture a smoothed energy landscape, which mitigates the phenomenon of mode collapse where the energy-based model (EBM) is less robust and capable of generating only a limited selection of protein sequences (e.g., those within the immediate vicinity of the known protein sequences in the data distribution). As described in more details below, during inference when the trained energy-based model (EBM) is applied to generate an output sequence by sampling from the data distribution, the trained energy-based model (EBM) may do so by “walking” the smoothed energy landscape of noisy protein sequences (e.g., a noisy data distribution), for example, through one or more iterations of gradient-based Markov Chain Monte Carlo (MCMC) sampling (e.g., Langevin Markov Chain Monte Carlo and/or the like), towards incrementally higher density regions of the data distribution and drawing a noisy output sequence therefrom before “jumping” to the true data distribution by denoising the noisy output sequence.

In some example embodiments, the protein design computation model may operate on a noisy embedding of protein sequences during training as well as inference. As noted, in some cases, the energy based model (EBM) may learn and sample from a data distribution of known protein sequences that have been adulterated with noise. The energy function of this data distribution may capture a smoothed energy landscape with less of the sharp gradient changes that limit the diversity of output protein sequences sampled from the data distribution. In some cases, each known protein sequence may be encoded to generate a corresponding sequence embedding before noise is added to each sequence embedding. For instance, in some cases, the energy based model (EBM) may be trained based on a noisy embedded training set of noisy embeddings of known protein sequences to learn a corresponding data distribution. During inference, a noisy sequence embedding may be sampled from the data distribution before being denoised and decoded to generate an output protein sequence. As described in more details below, the encoding of a protein sequence may include the addition of information, such as structural and/or environmental information for the protein sequence, to increase the semantic meaning of the resulting sequence embedding. Sampling from a noisy latent space occupied by noisy sequence embeddings may yield output protein sequences that are more likely to exhibit the desirable properties of the known protein sequences (e.g., drug-like properties such as binding affinity and specificity, stability, non-immunogenicity, human-ness, absence of self-association (or non-aggregation), lack of chemical liabilities (e.g., aspartate isomerization, oxidation, deamidation), and/or the like).

In some example embodiments, the encoding of a known protein sequence may project the known protein sequence from a sequence space (or discrete space) populated by protein sequences into a latent space populated by sequence embeddings, each of which being a latent space representation of a corresponding protein sequence. The sequence embedding of a known protein sequence may have a different dimensionality, or quantity of features, than the known protein sequence. For example, in instances where the encoding enriches the known protein sequence with information in addition to the identities and order of the constituent amino acid residues, the resulting sequence embedding may have a higher dimensionality (or a large quantity of features) than the known protein sequence. One example of additional information included in the sequence embedding is structural information indicative of the three-dimensional structure (or conformation) adopted by the known protein sequence. For instance, in some cases, the sequence embedding of the known protein sequence may include one or more structural tokens identifying, for each amino acid residue in the known protein sequence, one or more neighboring amino acid residue in three-dimensional space (e.g., one or more nearest amino acid residues, one or more amino acid residues within a threshold distance, and/or the like). It should be appreciated that in this context, the sequence embedding of a protein sequence may include a sequence of tokens. In addition to the aforementioned structural tokens, some tokens may encode (e.g., one-hot encoding and/or the like) the identity of each amino acid residue in the protein sequence and, in some cases, the sequential position of each amino acid residue.

In some example embodiments, the protein design computation model may generate an output sequence by at least sampling from the latent space, which may be smoothed with the addition of noise to sequence embeddings therein. Accordingly, in some cases, the trained energy-based model (EBM) may “walk” the smoothed latent space over one or more iterations of gradient-based Markov Chain Monte Carlo (MCMC) sampling (e.g., Langevin Markov Chain Monte Carlo and/or the like) to draw a noisy sequence embedding therefrom before “jumping” to the true latent space by denoising the noisy sequence embedding and returning to the sequence space by decoding the sequence embedding. The sequence (or primary structure) of a protein molecule alone may be insufficient to account for the presence (or absence) of certain desirable properties (e.g., drug-like properties such as binding affinity and specificity) at least because these properties may also be contingent upon the three-dimensional structure (e.g., secondary structure, tertiary structure, and/or the like) of the protein molecule. Enriching the sequence of a protein molecule with additional information, such as the aforementioned structural tokens, may increase the semantic meaning of the resulting sequence embedding by capturing at least some relationships between the sequence, conformation (or three-dimensional structure), and properties of the protein molecule. For example, the distance between two or more sequence embeddings in the corresponding latent space may reflect similarities (or dissimilarities) in protein sequence as well as conformation (or three-dimensional structure). As such, the latent space also exhibits greater continuity than the more sparsely populated sequence space. Sampling from the latent space may therefore yield output protein sequences that are diverse as well as more likely to exhibit the requisite combination of desirable properties (e.g., drug-like properties).

In some example embodiments, the protein design computation model may include multiple energy-based models (EBMs) trained in combination to learn different data distributions. For example, in some cases, the protein design computation model may include a first energy-based model (EBM) and a second energy-based model (EBM). In some cases, the first energy-based model may be trained to approximate a first data distribution of protein sequences while the second energy-based model may be trained to approximate a second data distribution of protein sequences. Furthermore, in some cases, the first energy-based model may be trained to approximate the first data distribution based on the gradient of the energy function associated with the second energy-based model, which quantities changes across the second data distribution. For example, in cases where the first data distribution may be associated with a more limited training set (e.g., with an inadequate quantity of known protein sequences) than the second data distribution, combining the training of the first energy-based model (EBM) and the second energy-based model (EBM) in this manner may enable the first energy-based model to learn from the larger training set of the second data distribution while avoiding the catastrophic forgetting the can occur when the first energy-based model is trained on both training sets. For instance, in some cases, the second data distribution may be associated with a larger set of known protein sequences (e.g., the Observed Antibody Space (OAS)) while the first data distribution may be associated with a smaller subset of known protein sequences that exhibit one or more desirable properties (e.g., antibodies binding to certain target molecules). In instances where the subset of known protein sequences contain relatively few known protein sequences, in addition to adjusting the parameters of the first energy-based model (EBM) to increase the similarity between the protein sequences generated by the first energy-based model (EBM) and the known protein sequences from the first data distribution, the parameters of the first energy-based model may be adjusted based on the gradient of the energy function associated with the second energy-based model. This energy function, which provides a density estimation of the second data distribution of protein sequences, may supplement the training of the first energy-based model by providing a surrogate density estimation for at least some of the regions in the first data distribution without adequate characterization by known protein sequences.

In some example embodiments, the energy-based model (EBM) may be trained to generate an output sequence having one or more desirable properties by at least applying, to an input sequence, one or more modifications. In some cases, the energy-based model (EBM) may be trained to learn the data distribution of the training set such that the modifications made to the input sequence are consistent with patterns of amino acid residues observed in the sample sequences. Examples of modifications that can be made to the input sequence may include changing the identity of one or more amino acid residues in the input sequence as well as changing the length of the input sequence through the insertion and/or deletion of one or more amino acid residues. It should be appreciated that the length of the input sequence may change frequently throughout the generative process as one or more amino acid residues may be inserted and/or deleted during each iteration of gradient based Markov Monte Carlo (MCMC) sampling. A conventional variable-length representation of the input sequence may require the energy-based model (EBM) to adjust to accommodate each length change, increasing the computational burden of the generative process. Accordingly, in some cases, the computational complexities that arise from the length of the input sequence changing during the generative process may be reduced by the energy-based model operating on a fixed-length representation of the input sequence instead of a conventional variable length representation of the input sequence. For example, the protein design engine may generate a fixed-length representation of the input sequence prior to generating the corresponding noisy sequence or, in some cases, a corresponding noisy sequence embedding. In some cases, the input sequence may be rendered in a fixed length representation by applying a structural role based numbering scheme in which each amino acid residue in the input sequence is assigned an integer position in a fixed length sequence (e.g., selected from a range of integers such as [1, 149]) corresponding to the structural role of the amino acid residue. A gap at any position in the fixed-length sequence where the input sequence lacks an amino acid residue having the corresponding structural role may be represented by a gap character such that each position in the fixed-length representation of the input sequence may be occupied by either an amino acid residue (e.g., one of twenty canonical amino acid residues) or a gap character. Moreover, an amino acid residue may be inserted into the input sequence by replacing a token encoding a gap character in the fixed-length representation of the input sequence with a token encoding the identity of the amino acid residue while an amino acid residue may be deleted from the input sequence by replacing a token encoding the identity of the amino acid residue in the fixed-length representation of the input sequence with a token encoding a gap character.

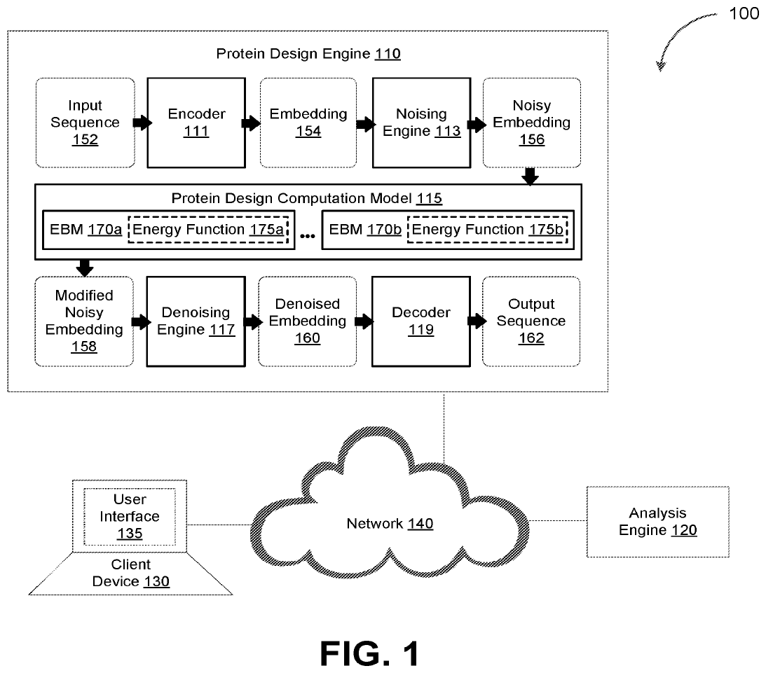

FIG. 1 depicts a system diagram illustrating an example of a protein design system 100, in accordance with some example embodiments. Referring to FIG. 1, the protein design system 100 may include a protein design engine 110, an analysis engine 120, and a client device 130. As shown in FIG. 1, the protein design engine 110, the analysis engine 120, and the client device 130 may be communicatively coupled via a network 140. The client device 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the protein design engine 110 may include an encoder 111, a noising engine 113, a protein design computation model 115, a denoising engine 117, and a decoder 119. In some cases, the protein design engine 110 may apply the protein design computation model 115 to generate, based at least on an input sequence 152, an output sequence 162. In the example shown in FIG. 1, the protein design computation model 115 may include one or more energy-based models 170 including, for example, a first energy-based model 170a, a second energy-based model 170b, and/or the like. In some cases, each of the one or more energy-based models 170 may be trained to approximate a corresponding data distribution. For example, in some cases, the first energy-based model 170a and the first energy function 175a parameterized by the parameters (e.g., weights, biases, and/or the like) of the first energy-based model 170a may approximate a first data distribution of protein sequences. The second energy-based model 170b and the second energy function 175b parameterized by the parameters (e.g., weights, biases, and/or the like) of the second energy-based model 170b may approximate a second data distribution of protein sequences. In instances where an inadequate quantity of known protein sequences characterizing the first data distribution are available to train the first energy-based model 170a, the first energy-based model 170a may be trained to approximate the first data distribution based on the first gradient of the first energy function 175a and the second gradient of the second energy function 175b. For instance, as described in more details below, the first energy-based model 170a may be trained to approximate the first data distribution by applying, to its parameters (e.g., weights, biases, and/or the like) of the first energy-based model 170a, one or more adjustments that increase (or maximize) a first similarity between a first output of the first energy-based model 170a and sample sequences from the first data distribution as well as a second similarity between a second output of the second energy-based model 170b and sample sequences from the second data distribution.

In some example embodiments, the protein design computation model 115 may generate the output sequence 162 by at least applying the first energy-based model 170a to modify the input sequence 152 to increase the likelihood of the output sequence 162 being in the first data distribution of protein sequences. In instances where the first data distribution of protein sequences exhibit one or more desirable properties (e.g., drug-like properties such as affinity, specificity, biological activity, developability, and/or the like), the output sequence 162 may be generated to also exhibit the one or more desirable properties. As described in more details below, the protein design computation model 115 may modify the input sequence 152 over one or more successive iterations of gradient-based Markov Chain Monte Carlo (MCMC) sampling (e.g., Markov Chain Monte Carlo (MCMC) with Langevin dynamics and/or the like). For example, in some cases, each iteration of gradient-based Markov Chain Monte Carlo (MCMC) sampling may include drawing, from the first data distribution, a sample that includes one or more modifications to the input sequence 152.

In some cases, the sampling from the first data distribution may be guided by the first energy function 175a. For example, samples drawn from low density regions of the first data distribution, which are populated by protein sequences without the one or more desirable properties, may be assigned a high energy value by the first energy function 175a to indicate a lower likelihood of being in the first data distribution. Contrastingly, samples drawn from high density regions of the first data distribution, which are populated by protein sequences with the one or more desirable properties, may be assigned a low energy value by the first energy function 175a to indicate a higher likelihood of being in the first data distribution. Accordingly, the gradient of the first energy function 175a, which corresponds to a change in energy values, may approximate a change in density across the first data distribution. Sampling from the first data distribution based on the gradient of the first energy function 175a may include drawing samples based on changes in the energy value assigned to each sample by the first energy function 175a. For instance, each subsequent iteration of gradient-based Markov Chain Monte Carlo (MCMC), samples may be drawn from incrementally higher density regions of the first data distribution, which are populated by protein sequences exhibiting the one or more desirable properties. The first energy function 175a may assign a corresponding lower energy value to those samples to indicate a higher likelihood of being in the first data distribution. It should be appreciated that it some cases, each iteration of gradient-based Markov Chain Monte Carlo (MCMC) may include further modifying one or more samples from a previous iteration determined to have a lower energy value than the other samples from that previous iteration.

In some example embodiments, instead of the first energy-based model 170a parameterizing the first energy function 175a, the first energy-based model 170a may parameterize a score function that outputs, for each sample drawn from the first data distribution, a score corresponding to the change in density observed at the location of the sample. In some cases, the score function may approximate the gradient of the first energy function 175a which, as noted, approximates a change in density across the first data distribution. As such, in some cases, the sampling from the first data distribution may be guided by the score function (instead of the first energy function 175a) such that each successive sample is drawn from incrementally higher density regions of the first data distribution. For example, the score function may assign a first score to a first sample indicating a more positive local change (e.g., an increase or a smaller decrease) in the density of the first data distribution at a first location of the first sample and a second score to a second sample indicating a less positive local change (e.g., a smaller increase or a decrease) in the density of the first data distribution at a second location of the second sample. In some cases, the first energy-based model 170a may draw a third sample from the first data distribution by further modifying the first sample in order to sample the third sample from a higher density region of the first data distribution than the first sample and the second sample.

In some example embodiments, overfitting of the protein design computation model 115 to the sample sequences in the training set may be avoided by the protein design computation model 115 operating on noisy protein sequences. For example, in some cases, the protein design computation model 115 may be trained based on a noisy training set of sample sequences, each of which being a known protein sequence that has been adulterated with noise (e.g., Gaussian noise such as isotropic Gaussian noise and/or the like). Furthermore, the protein design computation model 115 may generate the output sequence 162 by applying the first energy-based model 170a to modify a noisy embedding 156 of the input sequence 152. For instance, as shown in FIG. 1, the noising engine 113 may generate the noisy embedding 156 by at least adding noise (e.g., Gaussian noise such as isotropic Gaussian noise and/or the like) to the input sequence 152. The protein design computation model 115 may apply the first energy-based model 170a to modify the noisy embedding 156 of the input sequence 152, thus generating the modified noisy embedding 158. In some cases, the first energy-based model 170a may be applied to modify some portions of the noisy embedding 156 but not others. For instance, in some cases, the modification of the noisy embedding 156 may be limited to one or more adjustable segments of the input sequence 152, thus avoiding altering one or more fixed segments within the input sequence 152. Moreover, the denoising engine 117 may remove the noise present in the modified noisy embedding 158 to generate a denoised embedding 160 before the output sequence 162 is generated therefrom. As described in more details below, the first energy-based model 170a may generate the modified noisy embedding 156 by “walking” the smoothed energy landscape of noisy protein sequences before the denoising engine 117 denoises the modified noisy embedding 156, thus “jumping” back to the true data distribution.

In some example embodiments, the noisy embedding 156 of the input sequence 152 may be generated by the noising engine 113 adding noise (e.g., Gaussian noise such as isotropic Gaussian noise and/or the like) to an embedding 154 of the input sequence 152. In some cases, the embedding 154 may include additional information associated with the input sequence 152. For example, in some cases, the embedding 154 may be generated by the encoder 111 enriching (or upsampling) the input sequence 152 with structural information in the form of one or more structural tokens, each of which identifying the nearest neighboring amino acid residue in three-dimensional space of each amino acid residue in the input sequence 152. In doing so, the encoder 111 may map the input sequence 152 from a sparsely populated sequence space to a more continuous and semantically meaningful latent space from which the protein design computation model 115 samples the modified noisy embedding 158. For instance, the latent space may better capture the relationships between protein sequence, conformation (or three-dimensional structure), and properties, with the distance between two or more sequence embeddings in the latent space being reflective of the similarities (or dissimilarities) in protein sequence as well as conformation (or three-dimensional structure). Referring again to FIG. 1, in the example shown, the modified noisy embedding 158 generated by sampling from the latent space may be denoised by the denoising engine 117 before the resulting denoised embedding 160 is decoded, or mapped from the latent space to the sequence space, by the decoder 119. The output sequence 162 that is generated in this manner may be more likely to exhibit the one or more desirable properties of protein sequences in the first data distribution. However, it should be appreciated that instead of the encoder 111 generating the embedding 154 by enriching the input sequence 152 with additional information, the encoder 111 may implement an identity function, in which case the embedding 154 may be generated without the input sequence 152 being enriched with any additional information.

In some example embodiments, the protein design computation model 115 may generate the modified noisy embedding 158 by at least applying the first energy-based model 175a to modify the noisy embedding 156 of the input sequence 152. Examples of modifications may include changing the identity of one or more amino acid residues in the input sequence 152 as well as changing the length of the input sequence 152 through the insertion and/or deletion (or removal) of one or more amino acid residues. In cases where the first energy-based model 175a operates on a variable-length representation of the input sequence 152, the first energy-based model 175a may require adjustments to accommodate the changes in the length of the input sequence 152, which may occur frequently throughout the generative process as one or more amino acid residues are inserted and/or deleted (or removed) during each iteration of gradient based Markov Monte Carlo (MCMC) sampling. To avoid the computational complexities imposed by changes to the length of the input sequence 152, the first energy-based model 175a may operate on a fixed-length representation of the input sequence 152 instead of a variable-length representation of the input sequence 152. For example, in cases where the input sequence 152 is rendered in a fixed length representation by applying a structural role based numbering scheme in which each amino acid residue in the input sequence 152 is assigned an integer position in a fixed length sequence (e.g., selected from a range of integers such as [1, 149]) corresponding to the structural role of the amino acid residue, the embedding 154 and the noisy embedding 156 of the input sequence 152 may have a same length (e.g., same quantity of tokens) regardless of the quantity of amino acid residues forming the input sequence 152. As described in more details below, in instances where the input sequence 152 corresponds to an immunoglobulin protein (or an antibody), the aforementioned structural roles may correspond to the amino acid residue occupying a particular complementarity determining region (CDR) loop or one of the framework regions between a pair of complementarity determining region (CDR) loops. At any position of the embedding 154 and the noisy embedding 156 where the input sequence 152 lacks an amino acid residue having the corresponding structural role, the embedding 154 and the noisy embedding 156 of the input sequence 152 may include a gap character indicative of the absence of such amino acid residues.

Referring again to FIG. 1, the protein design computation model 115 may ingest the noisy embedding 156 of the input sequence 152 and generate the modified noisy embedding 158. As described in more details below, the denoising engine 117 may denoise the modified noisy embedding 158 to generate the denoised embedding 160 before the decoder 119 decodes the denoised embedding 119 to generate the output sequence 162. As noted, in some cases, the protein design computation model 115 may apply the first energy-based model 175a, which may generate the modified noisy embedding 158 by modifying the input sequence 152. In some cases, the first energy-based model 175a may modify the input sequence 152 by changing the identify of one or more of the amino acid residues in the input sequence 152. Alternatively and/or additionally, the first energy-based model 175a may modify the input sequence 152 by inserting and/or deleting (or removing) one or more amino acid residues in the input sequence 152. In instances where the input sequence 152 is rendered in the aforementioned fixed-length representation, the insertion of a particular type of amino acid residue at a certain position may be accomplished by replacing the corresponding gap character in the noisy embedding 156 of the input sequence 152 with that type of amino acid residue. Alternatively, the deletion (or removal) of an amino acid residue occupying a particular position in the input sequence 152 may be achieved by replacing the amino acid residue in the noisy embedding 156 of the input sequence 152 with a gap character.

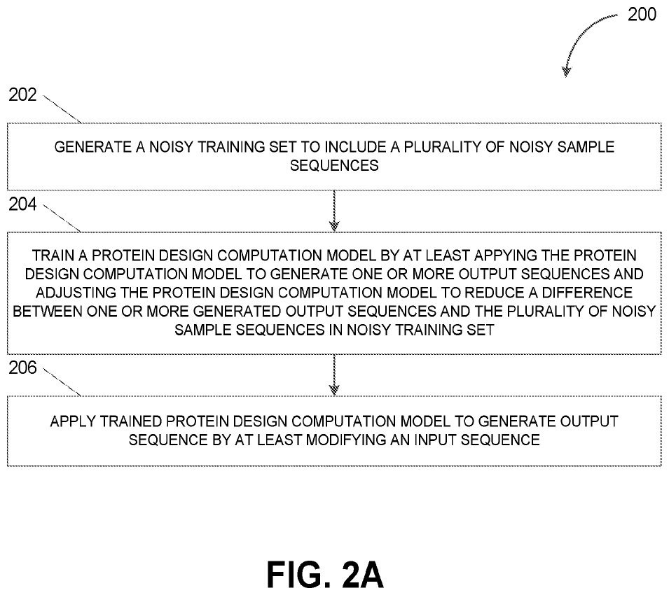

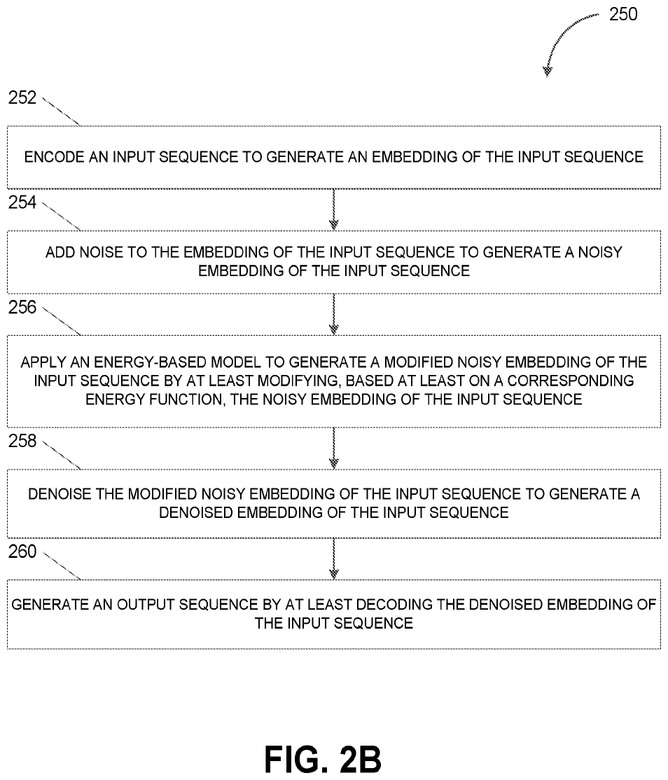

As noted, in some example embodiments, the protein design computation model 115 may operate on noisy protein sequences, which are protein sequences that have been adulterated with noise (e.g., Gaussian noise and/or the like). In the example shown in FIG. 1, the protein design computation model 115 may apply the first energy-based model 170a to modify the noisy embedding 156 of the input sequence 152 and generate the modified noisy embedding 158. In some cases, the noisy embedding 156 may be generated by the noisy engine 113 adding noise (e.g., Gaussian noise and/or the like) to the embedding 154 of the input sequence 152. It should be appreciated that while the embedding 154 can be enriched with additional information (e.g., structural information and/or the like), there may also be instances where the embedding 154 excludes additional information. Instead, the encoder 111 may implement an identity function, meaning that the embedding 154 may capture the same information present in the input sequence 152 including, for example, the identity of each amino acid residue, the sequential position of each amino acid residue, and/or the like. To further illustrate, FIG. 2A depicts a flowchart illustrating an example of a process 200 for computational protein design, in accordance with some example embodiments. Referring to FIGS. 1 and 2A, the process 200 may be performed by the protein design engine 110 to train and apply the protein design computation model 115 to generate the output sequence 162 by at least modifying the noisy embedding 156 of the input sequence 152. As described in more details below, the noisy embedding 156 of the input sequence 152 may be generated based on the embedding 154 of the input sequence 152, which may or may not be enriched with additional information (e.g., structural information and/or the like) associated with the input sequence 152.

At 202, the protein design engine 110 may generate a noisy training set to include a plurality of noisy sample sequences. In some example embodiments, to train the protein design computation model 115 to a data distribution populated by certain protein sequences such as protein sequences exhibiting one or more desirable properties (e.g., drug-like properties), the protein design engine 110 may generate a noisy training set containing a plurality of noisy sample sequences. Each noisy sample sequence in this case may be generated by adding noise (e.g., Gaussian noise such as isotropic Gaussian noise and/or the like) to a known protein sequence from the data distribution. Furthermore, in some cases, each noisy sample sequence may be a noisy sequence embedding that is generated by adding noise (e.g., Gaussian noise such as isotropic Gaussian noise and/or the like) to the embedding of the known protein sequence, which may or may not be enriched with additional information (e.g., structural information and/or the like. Training the protein design computation model 115 based on the noisy sample sequences in the noisy training set may mitigate the incidence of overfitting and mode collapse, which typically occur when the protein design computation model 115 is trained to approximate a high-dimensional data distribution (e.g., 20^{L} dimensions for length L protein sequences) based on disproportionately few known protein sequences.

In the example of the protein design computation model 115 shown in FIG. 1, the protein design computation model 115 may include the first energy-based model 170a. Training the protein design computation model 115 in this case may include training the first energy-based model 170a to approximate the data distribution based on the noisy sample sequences in the noisy training set. In some cases, the first energy-based model 170a may be a machine learning model, such as an artificial neural network (ANN), in which case the training of the first energy-based model 170a may include adjusting one or more parameters (e.g., weights, biases, and/or the like) of the machine learning model. Doing so may also determine the first energy function 175a, which is parametrized by the parameters of the first energy-based model 170a, to output an energy value corresponding to the likelihood of a protein sequence within the first data distribution. In some cases, the noisy training set may be applied to train the first energy-based model 175a in order to avoid overfitting the first energy-based model 175a, for example, to the few known protein sequences that are available to characterize the first data distribution. For example, in some cases, a known protein sequence, X, in R^{d} may be transformed into a noisy sample sequence by Y=X+N(0,σ²I_{d}).

In some cases, the noise level a may be determined based on the dimensionality and/or sparsity of the first data distribution in order to increase (or maximize) the quality of the noisy sample sequences in the noisy training set. For example, in some cases, the noise level σ may be set to approximately 0.5 (or another value). To further illustrate, consider the matrix X with entries χᵢᵢ, defined as follows

$${χ}_{{ii}^{′}} = \frac{‖ {X}_{i} - {X}_{{i}^{′}} ‖}{2 \sqrt{d}} ,$$

wherein d denotes the dimension of the data and

$$\frac{1}{2 \sqrt{d}}$$

is a scaling factor derived from the concentration of isotropic Gaussians in high dimensions. In some cases, the critical noise level σ_{c} may correspond to the largest entry in the matrix

$$χ ( e . g . , {σ}_{c} = {max}_{{ii}^{′}} {χ}_{i , {i}^{′}} )$$

such that the noisy sample sequences exhibit some degree of overlap for any noise level above the critical noise level (e.g., σ>σ_{c}).

In some example embodiments, the protein design engine 110 may generate each noisy sample sequence in the noisy training set by adding noise to a corresponding embedding of the sample sequence. For example, in some cases, the noising engine 113 may generate, based on a known protein sequence, a noisy sample sequence by at least adding noise (e.g., Gaussian noise such as isotropic Gaussian noise and/or the like) to an embedding of the known protein sequence. It should be appreciated that the embedding of the known protein sequence may or may not be enriched, for example, by the encoder 111, with additional information (e.g., structural information and/or the like). For instance, in cases where additional information is present, the embedding of the known protein sequence may include tokens encoding the identity, sequential position, and/or structural information of one or more amino acid residues in the known protein sequence.

In some cases, each noisy sample sequence in the noisy training set may be fixed in length, meaning that the length of each noisy sample sequence may be the same (e.g., same quantity of tokens) regardless of the quantity of amino acid residues in the corresponding known protein sequence. For example, in some cases, the encoder 111 of the protein design engine 110 may generate an embedding of a known protein sequence by applying a structural role based numbering scheme, which includes assigning, to each amino acid residue in the known protein sequence, an integer position in a fixed-length sequence (e.g., selected from a range of integers such as [1, 149]) corresponding to the structural role of the amino acid residue. In order to keep the length of the embedding the same regardless of the actual quantity of amino acid residues in the known protein sequence, the encoder 111 may insert a gap character at any position in the fixed-length sequence where the known protein sequence lacks an amino acid residue having the corresponding structural role. As noted, in some cases, the encoder 111 may further generate the embedding to include an encoding (e.g., one-hot encoding) of the identity of each amino acid residue in the known protein sequence and, in some cases, an encoding of the sequential position of each amino acid residue. To further illustrate, a known protein sequence may be represented by the embedding x=(x) (x₁, . . . , x_{d}), where each token x₁∈{1, . . . , 20, 21} indicates either the type of amino acid residue or a gap character at position l. In some cases, noise (e.g., Gaussian noise such as isotropic Gaussian noise and/or the like) may be added to the embedding x to generate a noisy embedding that is a numeric, floating point representation the original known protein sequence.

At 204, the protein design engine 110 may train the protein design computation model 115 by at least applying the protein design computation model 115 to generate one or more output sequences and adjusting the protein design computation model 115 to reduce a difference between the one or more generated output sequences and the plurality of noisy sample sequences in the noisy training set. In some example embodiments, the training of the protein design computation model 115 may include training, based at least on the noisy training set, the first energy-based model 170a to approximate the data distribution populated by certain protein sequences such as protein sequences that exhibit one or more desirable properties. In some cases, training the first energy-based model 170a may further include determining the corresponding first energy function 175a, which is parameterized by the parameters (e.g., weights, biases, and/or the like) of the first energy-based model 170a. For example, in some cases, the training of the first energy-based model 170a may include adjusting one or more parameters (e.g., weights, biases, and/or the like) of the first energy-based model 170a to reduce (or minimize) the difference between the output sequences generated by the first energy-based model 170a and the noisy sample sequences in the noisy training set. Doing so may also adjust the parameters of the first energy function 175a such that the first energy function 175a outputs a lower energy value for a first protein sequence that is within the data distribution than for a second protein sequence that is outside of the data distribution.

In some example embodiments, the protein design engine 110 may train the first energy-based model 170a by at least performing a gradient based Markov Chain Monte Carlo (MCMC) sampling, such as Markov Chain Monte Carlo (MCMC) sampling with Langevin dynamics and/or the like, to approximate the gradient of the first energy function 175a. In some cases, the gradient of the first energy function 175a may indicate changes in the density of the data distribution. For example, the gradient of the first energy function 175a may indicate transitions between different density regions of the data distribution including, for example, transitions between higher density regions and lower density regions of the data distribution. As will be described in more detail below, subsequent sampling from the data distribution may be guided by the first energy function 175a, in particular the gradient of the first energy function 175a, towards higher density regions of the data distribution, which are more likely to be populated by protein sequences exhibiting the one or more desirable properties. Moreover, in some cases, the gradient based Markov Chain Monte Carlo (MCMC) sampling to approximate the gradient of the first energy function 175a may include adjusting the parameters (e.g., weights, biases, and/or the like) of the first energy-based model 170a and that of the first energy function 175a over successive iterations to increase (or maximize) the similarity between the output sequences generated by the first energy-based model 170a and the noisy sample sequences in the noisy training set while reducing (or minimizing) the energy value determined by the first energy function 175a for these sequences.

To further illustrate, the training of the first energy-based model 170a may include learning the first energy function 170a, denoted as E_{θ}(x), which maps inputs, x, to a scalar “energy” value. The data distribution po (x) associated with the inputs x may be approximated by the Boltzmann distribution

$${p}_{θ} ( x ) ∝ {e}^{- {E}_{θ} ( x )} .$$

In some cases, the first energy-based model 170a may be trained via contrastive divergence with new sequences (or “samples”) being drawn from p_{θ}(x) by Markov-Chain Monte Carlo (MCMC) sampling. In the case of gradient based Markov Chain Monte Carlo sampling (e.g., Langevin Markov Chain Monte Carlo (MCMC) sampling), each sequence (or “sample”) may be initialized from a known protein sequence or a noise sequence before being refined with (discretized) Langevin diffusion

$${x}_{k + 1} = {x}_{k} - δ ∇ {f}_{θ} ( {x}_{k} ) + \sqrt{2 δ} {ε}_{k} , {ε}_{k} ∼ 𝒩 ( 0 , {I}_{d} )$$

wherein ∇ denotes the gradient of the first energy function 175a, k denotes the sampling iteration, δ is the (discretization) step size, and the noise εₖ is drawn from a normal distribution N at each iteration.

According to the foregoing formulation, the training of the first energy-based model 170a may include adjusting the parameters (e.g., weights, biases, and/or the like) of the first energy-based model 170a to increase (or maximize) the log-likelihood of the noisy sample sequences under the model. That is, the parameters (e.g., weights, biases, and/or the like) of the first energy-based model 170a may be adjusted to increase the likelihood of the first energy-based model 170a generating output sequences that are similar to the noisy sample sequences in the noisy training set. With this objective, the parameters of the first energy-based model 170a may be adjusted to decrease the energy of noisy training set, y⁻, while increasing the energy of noisy data sampled from the model, y⁺. That is, when trained, the first energy function 175a of the first energy-based model 170a may output a lower energy value for a first protein sequence that is within the data distribution (or sampled from a higher density region of the data distribution) than for a second protein sequence that is outside of the data distribution (or sampled from a lower density region of the data distribution). An additional ↑²-norm penalty may be added to the loss to regularize the energies.

$$arg max {E}_{y ∼ p} [ log log {p}_{θ} ( y ) ] = {arg max}_{θ} ( {E}_{{y}^{-} ∼ {p}_{θ}} [ {f}_{θ} ( {y}^{-} ) ] - {E}_{{y}^{+} ∼ p} [ {f}_{θ} ( {y}^{+} ) ] )$$

As noted, the first energy-based model 170a may be trained based on the noisy sample sequences in the noisy training set in order to avoid overfitting the first energy-based model 170a to the few known protein sequences characterizing the data distribution. In cases where few known protein sequences characterizing a high-dimensional (e.g., 20^{L} dimensions for length L protein sequences) data distribution are available, training the first energy-based model 170a based on the known protein sequences directly may yield a jagged energy landscape in which drastic changes in energy values are present between regions populated by the known protein sequences. Sampling from the data distribution based on the gradient of a jagged energy landscape may prevent an adequate exploration of the data distribution at least the steepness of the gradient may limit sampling to regions within the immediate vicinity of the known protein sequences. Contrastingly, training the first energy-based model 170a based on the noisy sample sequences may yield a smoothed energy landscape, with the gradient of the first energy function 175a being more gradual to enable a better exploration of the data distribution when sampling therefrom.

In some cases, when a known protein sequence X is transformed with additive noise (e.g., Gaussian noise) to yield the noisy sample sequence Y=X+ [symbol] (0,σ²I_{d}), the least-squares estimator of the known protein sequence X may be given by

$$\hat{x} ( y ) = y + {σ}^{2} ∇ p ( y ) ,$$

wherein p(y)=∫p(y|x)(p(x)dx is the probability distribution function of the smoothed density and gradients are with respect to the inputs, y, not the parameters of the first energy function 175a associated with the first energy-model 170a. In some cases, this estimator may be expressed in terms of g(y)=∇ log p(y), which is known as the score function parameterized by the first energy-based model 170a (e.g., the artificial neural network g_{ϕ}: [symbol] ^{d}→ [symbol] ^{d} implementing the first energy-based model 170a). Accordingly, the least-squares estimator may take the parametric form \hat{x}_{ϕ}(y)=y+σ²g_{ϕ}(y). Moreover, the foregoing formulation yields the learning objective below, which may be optimized with stochastic gradient descent without requiring Markov Chain Monte Carlo (MCMC) sampling.

$$ℒ ( ϕ ) = {𝔼}_{x ∼ p ( x ) , y ∼ p ( y \vert x )} {‖ x - {\hat{x}}_{ϕ} ( y ) ‖}^{2}$$

At 206, the protein design engine 110 may apply the trained protein design computation model 115 generate an output sequence by at least modifying an input sequence. In some example embodiments, the protein design computation model 115 may apply the first energy-based model 170a to generate the output sequence 162 by at least modifying the input sequence 152 while being guided by the first energy function 175a. For example, in some cases, the first energy-based model 170a may modify the noisy embedding 156 of the input sequence 152, which may be generated by the noising engine 113 adding noise (e.g., Gaussian noise such as isotropic Gaussian noise and/or the like) to the embedding 154 of the input sequence 152. Moreover, in some cases, the embedding 154 may be generated by the encoder 111 to include additional information, such as structural information and/or the like, associated with the input sequence 152.

In some cases, the first energy-based model 170a may modify the input sequence 152 by inserting, deleting (or removing), and/or changing the identity of one or more amino acid residues in the input sequence 152. In instances where the input sequence 152 is rendered in a fixed-length representation, for example, by the application of a structural role based numbering scheme, the deletion (or removal) of an amino acid residue may be achieved by replacing a token encoding the identity of the amino acid residue with a gap character while the insertion of an amino acid residue may be achieved by replacing a gap character with a token encoding the identity of the amino acid residue. The modifying of the input sequence 152 may be guided by the first energy function 175a (e.g., the gradient of the first energy function 175a). In particular, in some cases, the input sequence 152 may undergo successive iterations of modifications, each of which lowering the energy value of the input sequence 152.

For example, in some cases, the input sequence 152 may undergo a first modification and a second modification. Doing so may be tantamount to drawing, from the data distribution, a first sample and a second sample. In some cases, upon drawing the first sample and the second sample from the data distribution, the protein design computation model 115 may apply the first energy function 175a to determine an energy value indicative of the likelihood of each sample within the data distribution. A lower energy value in this case may indicate that the sample is drawn from a higher density region of the data distribution or, analogously, that the sample has a higher likelihood of being within the data distribution. As such, in some cases, upon drawing the first sample and the second sample, the protein design computation model 115 may apply the first energy-based model 170a to continue modifying the input sequence 152 and drawing additional samples from incrementally higher density regions of the data distribution until, for example, a sample exhibiting a threshold likelihood of being within the data distribution is drawn. For instance, in some cases, the first energy-based model 170a may be applied to further modify the input sequence 152 having the first modification instead of the second modification if the input sequence 152 having the first modification is assigned a lower energy value by the first energy function 175a. Doing so may be analogous to “walking” the energy landscape of the data distribution to sample from incrementally higher density regions of the data distribution. In instances where the first energy-based model 170a is modifying the noisy embedding 156 of the input sequence 152, the first energy-based model 170a may be operating in a noisy latent space in which the distance between two or more sequence embeddings therein is reflective of the similarities (or dissimilarities) in protein sequence as well as conformation (or three-dimensional structure). The energy landscape of the data distribution may be smoothed by the addition of noise, which reduces the sharp changes in the gradient of the first energy function 175a. Since the first energy-based model 170a is trained to approximate the data distribution of protein sequences exhibiting certain desirable properties (e.g., drug-like properties), the modifications made to the input sequence 152 may be consistent with the patterns of amino acid residues observed in the known protein sequences such that the same desirable properties are also present in the output sequence 162 generated therefrom.

FIG. 2B depicts a flowchart illustrating another example of a process 250 for protein design, in accordance with some example embodiments. Referring to FIGS. 1 and 2A-B, the process 250 may be performed by the protein design computation model 115 applied by the protein design engine 110, for example, to generate an output sequence based on an input sequence. In some cases, the process 250 may implement operation 206 of the process 200 shown in FIG. 2A.

At 252, the protein design engine 110 may encode an input sequence to generate an embedding of the input sequence. In some example embodiments, the encoder 111 may encode the input sequence 152 to generate the embedding 154 of the input sequence 152. The input sequence 152 may correspond to a known protein sequence or a noise sequence (e.g., a sequence of random amino acid residues). In some cases, the encoder 111 may encode the input sequence 152 by at least generating, for each amino acid residue in the input sequence 152, a token encoding the identity of each amino acid residue. In instances where the input sequence 152 is rendered in a fixed-length representation having the same quantity of tokens regardless of the quantity of amino acid residues in the input sequence 152, at least some of the tokens in the embedding 154 of the input sequence 152 may identify the type of amino acid residue or the gap character occupying the corresponding positions in the embedding 154 of the input sequence 152. For example, in some cases, the encoder 111 may generate the fixed-length representation of the input sequence 152 by applying a structural role based numbering scheme. Doing so may include aligning the amino acid residues forming the input sequence 152 to a fixed set of structural roles (e.g., corresponding to various complementarity determining region (CDR) loops or the framework regions therebetween) and inserting gap characters where the alignment indicates the absence of amino acid residues having certain structural roles. Accordingly, where there are a d quantity of possible structural roles, the resulting embedding 154 may include a series of tokens x=(x) (x₁, . . . , x_{d}), wherein each token xₗ∈{1, . . . , 20, 21} indicates either the type of amino acid residue or a gap character occupying position l. Moreover, in some cases, each token xₗ∈{1, . . . , 20, 21} may be generated to include a positional encoding to indicate the sequential position of the token at position I relative to the other tokens in the embedding 154.

In some example embodiments, the encoder 111 may generate the embedding 154 of the input sequence 152 with or without enriching the embedding 154 with additional information. In some cases, the encoder 111 may implement an identity function, meaning that the embedding 154 may include the same information present in the input sequence 152 including, for example, the identity of each amino acid residue, the sequential position of each amino acid residue, and/or the like. Alternatively, in instances where the embedding 154 is generated to include additional information, this additional may include, for example, structural information, environmental information, and/or the like. The addition of information may be tantamount to mapping the input sequence 152 from a sequence space (or discrete space) populated by protein sequences into a continuous latent space populated by sequence embeddings, each of which being a latent space representation of a corresponding protein sequence. For example, in some cases, the encoder 111 may generate the embedding 154 of the input sequence 152 to include one or more structural tokens. In some cases, the one or more structural tokens may describe the conformation (or three-dimensional structure) adopted by the input sequence 152. For instance, in some cases, a structural token may identify, for a corresponding amino acid residue in the input sequence 152, one or more nearest neighboring amino acid residue in three-dimensional space.

It should be appreciated that these structural tokens convey a different type of information than positional encoding. That is, instead of amino acid residues that are adjacent in the primary structure of the input sequence 152, the structural tokens identify amino acid residues that become adjacent through the folding of the input sequence 152. The presence structural information may increase the semantic meaning of the embedding 154. In instances where the properties of the input sequence 152 are contingent upon the conformation (or three-dimensional structure) adopted by the input sequence 152, incorporating structural information may improve the outcome of the subsequent generative process at least because the protein design computation model 115 is able to take into account at least some of the relationships that exist between the sequence, conformation (or three-dimensional structure), and properties of the input sequence 152.

At 254, the protein design engine 110 may add noise to the embedding of the input sequence to generate a noisy embedding of the input sequence. In some example embodiments, the noising engine 113 of the protein design engine 110 may generate, based at least on the embedding 154, the noisy embedding 156 for ingestion by the protein design computation model 115 (e.g., the first energy-based model 170a). For example, in some cases, the noising engine 113 may add, to the embedding 154, noise (e.g., Gaussian noise such as isotropic Gaussian noise and/or the like) in order to generate the noisy embedding 156 of the input sequence 152. As noted, in some cases, the protein design computation model 115 (e.g., the first energy-based model 170a) may be trained, based on a noisy training set of noisy sample sequences generated from known protein sequence exhibiting certain desirable properties, to approximate a noisy data distribution of protein sequences having the desirable properties. This noisy data distribution may exhibit a smoothed energy landscape with gradual gradient changes, which facilitates subsequent sampling (e.g., gradient-based Markov Chain Monte Carlo (MCMC) sampling and/or the like) therefrom. Contrastingly, in cases where the protein design computation model 115 (e.g., the first energy-based model 170a) is trained based on known protein sequences directly, the protein design computation model 115 (e.g., the first energy-based model 170a) may learn a jagged energy landscape in which drastic changes in energy values are present between regions populated by the known protein sequences. Unlike the gradual gradient of the noisy data distribution, the steep gradient of this jagged energy landscape may prevent an adequate exploration of the data distribution during the generative process at least because sampling may be confined to regions within the immediate vicinity of the known protein sequences. As described in more details below, by operating on the noisy embedding 156 of the input sequence 152, the protein design computation model 115 (e.g., the first energy-based model 170a) may “walk” the smoothed energy landscape of the noisy data distribution to sample from incrementally higher density regions of the noisy data distribution before “jumping” back to the true data distribution when a sample exhibiting a threshold likelihood of being within the noisy data distribution is drawn.

At 256, the protein design computation model 115 may apply an energy-based model (EBM) to generate a modified noisy embedding of the input sequence by at least modifying, based at least on a corresponding energy function, the noisy embedding of the input sequence. In some example embodiments, the protein design computation model 115 may apply the first energy-based model 170a to modify the noisy embedding 156 of the input sequence and generate the modified noisy embedding 158. In some cases, the first energy-based model 170a may modify the noisy embedding 158 of the input sequence 152 by inserting an amino acid residue, deleting (or removing) an amino acid residue, and/or changing an identity of an amino acid residue in the input sequence 152. As noted, the insertion or deletion (or removal) of an amino acid residue at a certain position in the input sequence 152 may be achieved without changing the length of the input sequence 152 by swapping out or in a token representative of a gap character.

In some example embodiments, the protein design computation model 115 may apply the first energy-based model 170a to modify the noisy embedding 158 of the input sequence 152 based on the first energy function 175a of the first energy-based model 170a. In some cases, the noisy embedding 156 may be modified to achieve lower energy configurations, which are tantamount to samples drawn from higher density regions of the noisy data distribution, as indicated by the energy value output by the first energy function 175a. In some cases, the protein design computation model 115 may perform a gradient based Markov Chain Monte Carlo (MCMC) sampling (e.g., Langevin Markov Chain Monte Carlo (MCMC) sampling) of the noisy data distribution in which the noisy embedding 156 of the input sequence 152 is modified over multiple successive iterations, with each iteration sampling from an incrementally higher density region of the noisy data distribution to increase the likelihood of the resulting modified noisy embedding 158 being in the noisy data distribution. Moreover, in some cases, the modifications made to the noisy embedding 156 of the input sequence 152 may be cumulative over the multiple successive iterations. For example, in some cases, the noisy embedding 156 of the input sequence 152 may undergo a first modification and a second modification. The protein design computation model 115 may apply the first energy function 175a to determine a first energy value of the noisy embedding 156 having the first modification and a second energy value of the noisy embedding 156 having the second modification. For a subsequent iteration of gradient-based Markov Chain Monte Carlo (MCMC) sampling, the first energy-based model 170a may be applied to further modify the noisy embedding 156 having the first modification if the first energy value is lower than the second energy value, indicating that the noisy embedding 156 having the first modification is sampled from a higher density region of the noisy data distribution and exhibits a higher likelihood of being within the noisy data distribution. In some cases, one or more additional iterations of the gradient-based Markov Chain Monte Carlo (MCMC) sampling may be performed, with the protein design computation model 115 applying the first energy-based model 170a to further modify the noisy embedding 156 of the input sequence 152, until one or more criteria are met. For instance, in some cases, the protein design computation model 115 may perform one or more additional iterations of gradient based Markov Chain Monte Carlo (MCMC) sampling until a threshold quantity of iterations are performed. Alternatively and/or additionally, the protein design computation model 115 may perform one or more additional iterations of gradient based Markov Chain Monte Carlo (MCMC) sampling until the energy value of the modified noisy embedding 156 or the likelihood of the modified noisy embedding 156 being within the noisy data distribution satisfy one or more thresholds.

At 258, the protein design engine 110 may denoise the modified noisy embedding of the input sequence to generate a denoised embedding of the input sequence. In some example embodiments, the denoising engine 117 of the protein design computation model 115 may generate the denoised embedding 160 by at least denoising the modified noisy embedding 158 generated by the protein design computation model 115 (e.g., the first energy-based model 170a). In some cases, the denoising engine 117 may include one or more machine learning models (e.g., transformer and/or the like) trained to denoise the modified noisy embedding 158 and recover the denoised embedding 160 therefrom. For example, in some cases, the one or more machine learning models may be trained based on the noisy training set to recover, for each noisy sample sequences in the noisy training set, the corresponding known protein sequence.

To further illustrate, as noted, a known protein sequence, X, in R^{d} may be transformed into a noisy sample sequence with the addition of noise to yield the noisy sample sequence Y=X+N(0,σ²I_{d}). Accordingly, in some cases, the denoising engine 117 may denoise the noisy modified embedding 158 generated by the protein design computation model 115 (e.g., the first energy-based model 170a) based at least on the least-squares estimator of the sample sequence X, which may be given by

$$\hat{x} ( y ) = y + {σ}^{2} ∇ p ( y ) ,$$

wherein p(y) is the probability distribution function of the smoothed density and gradients are with respect to the inputs, y, not the parameters of the first energy function 175a associated with the first energy-model 170a. This formulation defines the following loss function

$$ℒ ( ϕ ) = {𝔼}_{x ∼ p ( x ) , y ∼ p ( y \vert x )} {‖ x - {\hat{x}}_{ϕ} ( y ) ‖}^{2} ,$$

wherein ϕ:R^{d}→R denotes an artificial neural network (ANN) having parameters θ that implements the first energy-based model 170a and parameterizes the first energy function 175b trained to approximate the noisy data distribution of the noisy sample sequences Y. The noisy modified embedding 168 as well as any intermediate modified sequences at timestep τ, y_{τ}, may be drawn from the density

$$\frac{{e}^{- ϕ}}{Z}$$

in which Z is the partition function, an unknown normalization constant) via “walking” the smoothed energy landscape of the noisy data distribution approximated by the first energy-based model 170a with gradient-based Markov Chain Monte Carlo (e.g., Langevin Markov Chain Monte Carlo and/or the like) sampling. With the denoising performed by the denoising engine 117, the denoised embedding 160 corresponding to the noisy modified embedding 158 may be obtained from the true data distribution (e.g., the manifold M) by “jumping” back to the true data distribution (e.g., the manifold M) with the least-squares estimator

$$\hat{x} ( {y}_{r} - {σ}^{2} ∇ ϕ ( {y}_{r} ) ) .$$

Doing so amounts to approximating the score function, ψ, with the gradient of the first energy function 175a, such that ψ=∇ log log ƒ≈−∇ϕ. That is, the score function ψ may output a score corresponding to the gradient of the log-likelihood of the input sequence 152, which in turn approximates the gradient of the first energy function 175a. Moreover, in instances where the denoised embedding 160 does not include additional information (e.g., structural tokens and/or the like) and populates the corresponding latent space, the output sequence 162 may be generated directly therefrom (e.g., without further decoding by the decoder 119), for example, by recovering the output sequence 162 from the tokens in the denoised embedding 160 that encode the identities and, in some cases, the sequential positions, of the constituent amino acid residues. For example, in cases where the tokens in the denoised embedding 160 includes a one-hot encoding of the identities of individual amino acid residues or, in some cases, gap characters occupying each position within the denoised embedding 160, the output sequence 162 may be recovered with the application of an argmax operation before removing any gap characters.

At 260, the protein design engine 110 may generate an output sequence by at least decoding the denoised embedding of the input sequence. In some example embodiments, the decoder 119 of the protein design engine 110 may generate the output sequence 162 by at least decoding the denoised embedding 160 generated by the denoising engine 117. As noted, in some case, the protein design computation model 115 (e.g., the first energy-based model 170a) may operate in a noisy latent space to generate the modified noisy embedding 158 in cases where the noisy embedding 156 of the input sequence 152 incorporates additional information (e.g., structural tokens and/or the like). Accordingly, in some cases, in addition to the denoising of the modified noisy embedding 158, the resulting denoised embedding 160 may be decoded by the decoder 119 in order to generate the output sequence 162. For example, in some cases, the decoding of the denoised embedding 160 may include determining, based at least on the tokens in the denoised embedding 160, the identities and the sequential positions of the amino acid residues forming the output sequence 162.

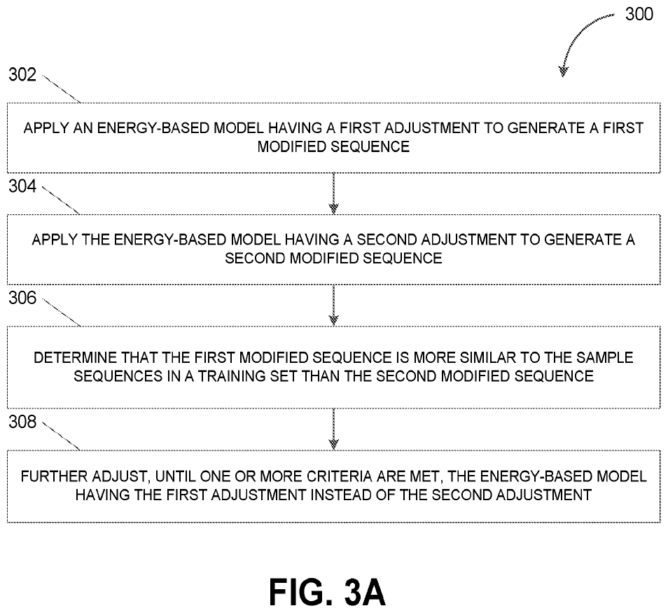

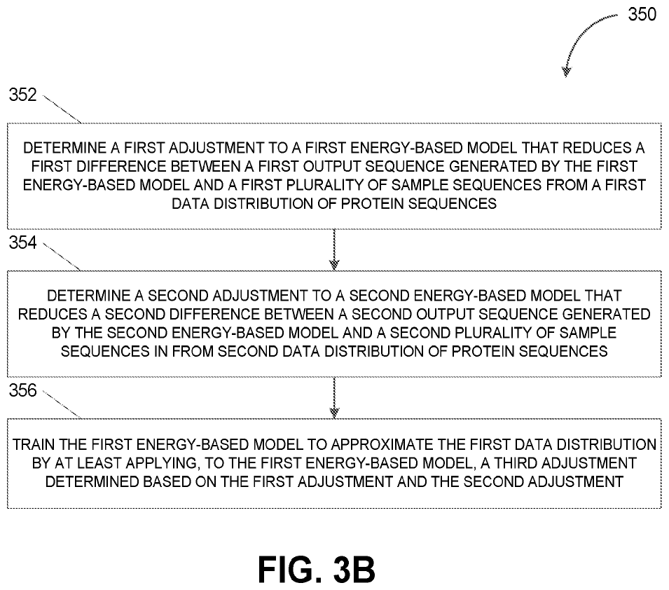

FIG. 3A depicts a flowchart illustrating an example of a process 300 for training the protein design computation model 115, in accordance with some example embodiments. Referring to FIGS. 1, 2A, and 3A, the process 300 may be performed by the protein design engine 110 to train the protein design computation model 115 such as, for example, each of the first energy-based model 170a and the second energy-based model 170b. As described in more details below, in some cases, the protein design computation model 115 may be trained through gradient based Markov Chain Monte Carlo (MCMC) sampling including, for example, Markov Chain Monte Carlo (MCMC) sampling with Langevin dynamics and/or the like). Moreover, in some cases, the process 300 may implement operation 204 of the process 200 shown in FIG. 2A.

At 302, the protein design engine 110 may apply an energy-based model (EBM) model to generate a first modified sequence. In some example embodiments, the protein design engine 110 may train the protein design computation model 115 including, for example, the first energy-based model 170a to approximate the data distribution of protein sequences exhibiting one or more desirable properties such that additional protein sequences exhibiting the same desirable properties can be generated by sampling therefrom. In some cases, the first energy-based model 170a may be trained to approximate the aforementioned data distribution based on a training set of sample sequences, each of which being a known protein sequence from the data distribution. In some cases, instead of being trained on the known protein sequences directly, the first energy-based model 170a may be trained based on noisy embeddings of the known protein sequences. That is, in some cases, the first energy-based model 170a may be trained based on a noisy training set of noisy sample sequences, each of which being an embedding of a known protein sequence that has been adulterated with noise (e.g., Gaussian noise such as isotropic Gaussian noise and/or the like).

In some example embodiments, the training of the first energy-based model 170a may include applying the first energy-based model 170a to modify an initial sequence (e.g., a known protein sequence or a noise sequence) and adjusting the parameters (e.g., weights, biases and/or the like) of the first energy-based model 170a to increase, for example, incrementally over multiple successive iterations, the similarity between the resulting modified sequences and the noisy sample sequences in the noisy training set. In some cases, the parameters (e.g., weights, biases, and/or the like) of first energy-based model 170a may undergo different adjustments before further adjustments are made to the adjustment that yielded protein sequences that are more similar to the noisy sample sequences in the noisy training set. For example, in some cases, a first adjustment may be made to the parameters of the first energy-based model 170a before the first energy-based model 170a having the first adjustment is applied to modify an input sequence and generate at least a first modified sequence. As described in more details below, the first energy-based model 170a having a second adjustment may be applied to generate at least a second modified sequence before further adjustments are made to the first energy-based model 170a having either the first adjustment or the second adjustment. Doing so may train the first energy-based model 170a to approximate a noisy data distribution populating a continuous latent space which, as noted, may facilitate subsequent sampling (e.g., gradient-based Markov Chain Monte Carlo (MCMC) sampling and/or the like).

In some example embodiments, the training of the first energy-based model 170a may further include determining the first energy function 175a. As noted, in some cases, the first energy function 175a may be parameterized by the parameters (e.g., weights, biases, and/or the like) of the first energy-based model 170a. Accordingly, in some cases, training the first energy-based model 170a, which includes adjusting the parameters of the first energy-based model 170a, may also include adjusting the parameters of the first energy function 175a. For example, in some cases, the first energy function 175a may be determined by performing gradient-based Markov Chain Monte Carlo (MCMC) sampling (e.g., Langevin Markov Chain Monte Carlo (MCMC) sampling and/or the like) to approximate the gradient of the noisy data distribution. Doing so may include adjusting, over multiple successive iterations, the parameters of the first energy function 175a such that the first energy function 175a assigns a lower energy value to a first sequence that is more similar to the noisy sample sequences in the noisy training set than to a second sequence that is less similar to the noisy sample sequences in the training set. Once the first energy-based model 175a is trained, the first energy function 175a may output energy values that differentiate between protein sequences sampled from higher density regions of the noisy data distribution and those sampled from lower density regions of the noisy data distribution.

At 304, the protein design engine 110 may apply the energy-based model having a second adjustment to generate a second modified sequence. In some example embodiments, upon applying the first energy-based model 170a having the first adjustment to generate at least the first modified sequence, the protein design engine 110 may apply the first energy-based model 170a having a second adjustment to generate at least a second modified sequence. It should be appreciated that the first adjustment and the second adjustment may include different changes to the parameters (e.g., weights, biases, and/or the like) of the first energy-based model 170a. As such, applying the first energy-based model 170a having the second adjustment to modify the input sequence may yield different modified sequences than applying the first energy-based model 170a to modify the same input sequence.

At 306, the protein design engine 110 may determine that the first modified sequence is more similar to the sample sequences in a training set than the second modified sequence. In some example embodiments, the protein design engine 110 may select, for further adjustments during a subsequent iteration, the first energy-based model 170a having the first adjustment instead of the first energy-based model 170b having the second adjustment if the modified sequences generated by the first energy-based model 170a having the first adjustment is more similar to the sample sequences in the training set or, in some cases, the noisy sample sequences in the noisy training set. That the first modified sequence is more similar to the sample sequences in the training set (or the noisy sample sequences in the noisy training set) than the second modified sequence may indicate that the first energy-based model 170a having the first adjustment better approximates the data distribution of the sample sequences (or noisy sample sequences) than the second energy-based model 170a having the second adjustment. In some cases, the similarity between a modified sequence generated by the first energy-based model 170a and the sample sequences in the training set (or the noisy sample sequences in the noisy training set) may be quantified by a similarity metric. Examples of the similarity metric include an antibody likeness metric (e.g., biophysical properties such as molecular weight, length, hydrophobicity, hydrophilicity, and/or the like), sequence similarity (e.g., edit distance and/or the like), a naturalness metric (e.g., likelihood under a pre-trained protein language model), and/or the like. In some cases, the protein design engine 110 may select, based at least on the first modified sequence having a higher similarity metric than the second modified sequence, the first energy-based model 170a having the first adjustment instead of the first energy-based model 170a having the second adjustment to undergo one or more additional iterations of adjustments.

At 308, the protein design engine 110 may further adjust, until one or more criteria are met, the energy-based model having the first adjustment instead of the second adjustment. In some example embodiments, the protein design engine 110 may further adjust the first energy-based model 170a having the first adjustment instead of the first energy-based model 170b having the second adjustment in instances where the first modified sequence generated by the first energy-based model 170a having the first adjustment is more similar to the sample sequences in the training set (or the noisy sample sequences in the noisy training set) than the second modified sequence generated by the first energy-based model 170a having the second adjustment. For example, during a subsequent iteration of adjustments, the protein design engine 110 may make further adjustments to the parameters (e.g., weights, biases, and/or the like) the first energy-based model 150a having the first adjustments before applying the further adjusted first energy-based model 150a to generate one or more additional modified sequences. In some cases, the first energy-based model 170a may be further adjusted in order to further increase the similarity between the modified sequences output by the first energy-based model 170a and the sample sequences in the training set (or the noisy sample sequences in the noisy training set). In some cases, the protein design engine 110 may continue to adjust the first energy-based model 170a until one or more criteria are satisfied. For instance, in some cases, the protein design engine 110 may continue to adjust the parameters (e.g., weights, biases, and/or the like) of first energy-based model 170a until the protein design engine 110 has performed a threshold quantity of iterations of adjustments. Alternatively and/or additionally, the protein design engine 110 may continue to adjust the parameters (e.g., weights, biases, and/or the like) of first energy-based model 170a until the similarity (or similarity metric) between the modified sequences generated by the first energy-based model 170a and the sample sequences in the training set (or the noisy sample sequences in the noisy training set) satisfies one or more thresholds.

FIG. 3B depicts a flowchart illustrating another example of a process 350 for training the protein design computation model 115, in accordance with some example embodiments. Referring to FIGS. 1, 2A, and 3B, the process 350 may be performed by the protein design engine 110 to train the protein design computation model 115 including, for example, the first energy-based model 170a, the second energy-based model 170b, and/or the like. In some example embodiments, the process 350 may be performed in order to train the first energy-based model 170a to approximate a first data distribution of protein sequences based on the gradient of a second data distribution of protein sequences. For example, in some cases, the process 350 may be performed in instances where too few known protein sequences characterizing the first data distribution are available for training the first energy-based model 170a. As described in more details below, in some cases, the second energy-based model 170b may be trained to approximate the second data distribution of protein sequences such that the second energy function 175b may be applied to provide additional guidance while the first energy-based model 170a is trained to approximate the first data distribution through, for example, gradient based Markov Chain Monte Carlo sampling (e.g., Langevin Markov Chain Monte Carlo and/or the like) across multiple data distributions. In some cases, the process 350 may implement operation 204 of the process 200 shown in FIG. 2A.

At 352, the protein design engine 110 may determine a first adjustment to a first energy-based model that reduces a first difference between a first output sequence generated by the first energy-based model and a first plurality of sample sequences from a first data distribution of protein sequences. In some example embodiments, the protein design engine 110 may combine the training of multiple energy-based models including, for example, the first energy-based model 170a and the second energy-based model 170b. For example, in some cases, the training of the first energy-based model 170a to approximate the first data distribution of protein sequences may be combined with the training of the second energy-based model 170b to approximate the second data distribution in instances where an inadequate quantity of known protein sequences from the first data distribution are available for training the first energy-based model 170a. Accordingly, in some cases, the protein design engine 110 may determine, for the first energy-based model 170a, a first adjustment that increases the similarity (or similarity metric) between the output sequences generated by the first energy-based model 170a and the sample sequences from the first data distribution (e.g., noisy sequence embeddings from a noisy data distribution). However, as will be described in more detail below, instead of applying the first adjustment directly to the parameters (e.g., weights, biases, and/or the like) of the first energy-based model 170a, the protein design engine 110 may further determine the adjustments made to the parameters (e.g., weights, biases, and/or the like) of the first energy-based model 170a based on the gradient of the second energy function 175b of the second energy-based model 170b trained to approximate the second data distribution of protein sequences.

At 354, the protein design engine 110 may determine a second adjustment to a second energy-based model that reduces a difference between a second output sequence generated by the second energy-based model and a second plurality of sample sequences from a second data distribution. In some example embodiments, the protein design engine 110 may determine a second adjustment to the parameters (e.g., weights, biases, and/or the like) of the second energy-based model 170b to increase the similarity between one or more output sequences generated by the second energy-based model 170b and the sample sequences from the second data distribution of protein sequences (e.g., noisy sequence embeddings from a noisy data distribution). As noted, an inadequate quantity of known protein sequences from the first data distribution may be available to train the first energy-based model 170a to approximate the first data distribution but a larger quantity of known protein sequences from the second data distribution may be available for training the second energy-based model 170b to approximate the second data distribution. As such, in some cases, the density of at least some regions in the first data distribution may be indeterminate due to the lack of known protein sequences populating those regions. In those regions of the first data distribution where the density of the first data distribution cannot be determined due to the lack of known protein sequences populating these regions, the gradient of the second energy function 175b may provide a surrogate density estimation. Thus, combining the training of the first energy-based model 170a and the second energy-based model 170a may improve the performance of the first energy-based model 170a by at least increasing the precision and accuracy of the approximation of the first data distribution.

At 356, the protein design engine 110 may train the first energy-based model by at least applying, to the first energy-based model, a third adjustment determined based on the first adjustment and the second adjustment. In some example embodiments, the protein design engine 110 may determine, based at least on the first adjustment and the second adjustment, a third adjustment to apply to the parameters (e.g., weights, biases, and/or the like) of the first energy-based model 170a. For example, in some cases, the third adjustment may be a sum of the first adjustment and the second adjustment. Alternatively, the third adjustment may be weighted sum in which the first adjustment and the second adjustment are associated with different weights. The training of the first energy-based model 170a may include applying, to the parameters (e.g., weights, biases and/or the like) of the first energy-based model 170a, the third adjustment. In some cases, the third adjustment may capture an estimate of the density across the first data distribution of protein sequences and the second data distribution of protein sequences, including those regions of the first data distribution where the density of the first data distribution is indeterminate due to the lack of known protein sequences populating those regions. Accordingly, applying the third adjustment to the first energy-based model 170a may enable the first energy-based model 170a to better approximate the first data distribution despite the lack of known protein sequences characterizing at least some regions of the first data distribution.

As noted, in some example embodiments, the first energy-based model 170a may be trained to approximate and subsequently sample from a noisy data distribution of noisy protein sequences instead of the true data distribution of protein sequences that have not been perturbed with any noise. Training the first energy-based model 170a to approximate a data distribution of protein sequences, such as the data distribution of protein sequences exhibiting certain desirable properties (e.g., drug-like properties), may include determining the first energy function 170a such that the first energy function 170a assigns a lower energy value to protein sequences sampled by higher density regions of the first data distribution than to those sampled from lower density regions of the first data distribution. Moreover, the gradient of the first energy function 170a may approximate the changes in density across the first data distribution.

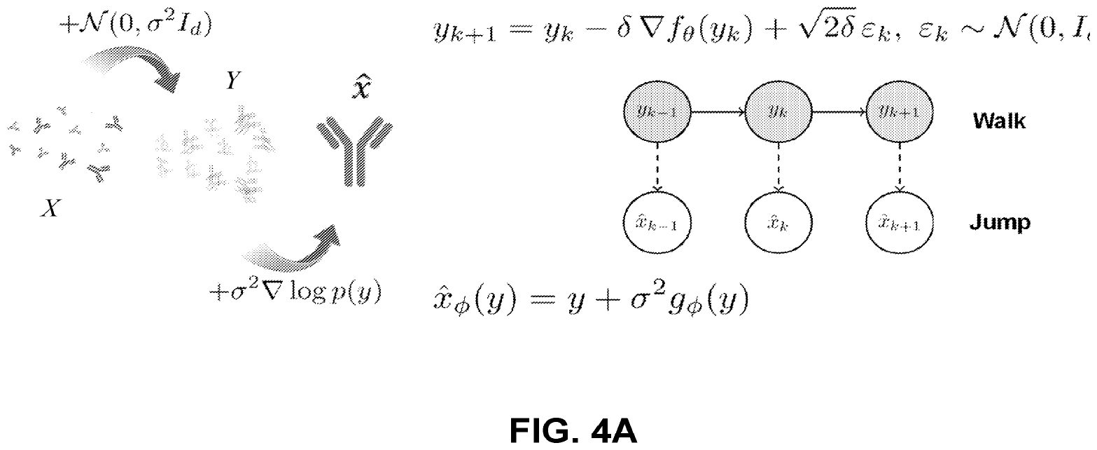

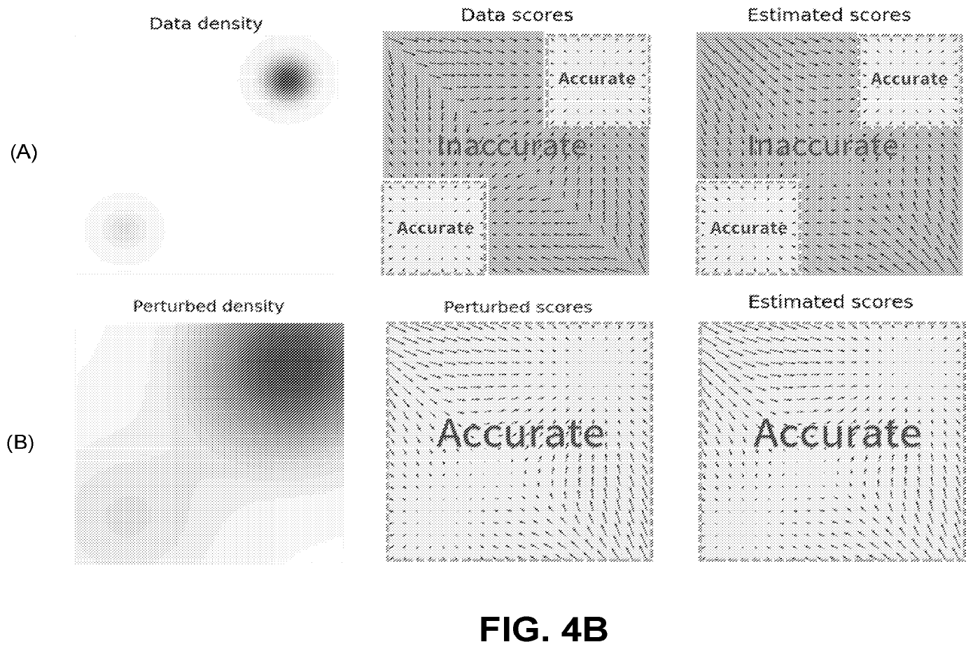

To further illustrate, FIG. 4A depicts a schematic diagram illustrating an example of a sampling from a noisy data distribution, in accordance with some example embodiments. As shown in FIG. 4A, known protein sequences X may be transformed into noisy sequences Y with the addition of noise [symbol] (0,σ²I_{d}). The addition of the noise [symbol] (0,σ²I_{d}) may project the known protein sequences X into a noisy data distribution populated by the noisy sequences Y, which exhibits a smoother energy landscape than the data distribution populated by the known protein sequences X. In some cases, the first energy-based model 170a may sample from the noisy data distribution, which includes “walking” its energy landscape towards incrementally higher density regions of the noisy data distribution populated protein sequences exhibiting the desirable properties. For example, FIG. 4A shows that the “walk” across the energy landscape include drawing samples yₖ₋₁ at sampling iteration k−1, yₖ at sampling iteration k, and yₖ₊₁ at sampling iteration k+1. In some cases, the “walk” across the energy landscape of the noisy data distribution may be guided by the gradient of the first energy function 175a such that the energy value of sample yₖ is lower than the energy value of the sample yₖ₋₁ and the energy value of the sample yₖ₊₁ is lower still than that of the sample yₖ. Moreover, each sampling iteration may include further modifying the sample drawn during a previous iteration. Accordingly, as shown below, the sample yₖ₊₁ drawn from the noisy data distribution during sampling iteration k+1 may be generated based on the sample yₖ drawn during the previous sampling iteration k, with the noise εₖ being drawn from the normal distribution [symbol] at each sampling iteration.

$${y}_{k + 1} = {y}_{k} - δ ∇ {f}_{θ} ( {y}_{k} ) + \sqrt{2 δ} {ε}_{k} , {ε}_{k} ∼ 𝒩 ( 0 , {I}_{d} )$$

Referring again to FIG. 4A, in some cases, the protein sequence x may be generated when a corresponding noisy protein sequence y drawn from noisy data distribution is denoised and projected back to the true data distribution by the denoising engine 117 applying the least squares estimator σ²∇p(y) (e.g., \hat{x}_{ϕ}(y)=y+σ²g_{ϕ}(y)). This constitutes the “jump” shown in FIG. A. Furthermore, in the example shown in FIG. 4, a “jump” back to the true data distribution may be performed at each sampling iteration while the first energy-based model 170a “walks” the energy landscape of the noisy data distribution and draws samples therefrom. For example, the protein sequence \hat{x}ₖ₋₁ may be generated when the sample yₖ₊₁ drawn from the noisy data distribution during sampling iteration k+1 is denoised and projected back to the true data distribution while the protein sequence xₖ may be generated when the sample yₖ drawn from the noisy data distribution during the subsequent sampling iteration k+1 is denoised and projected back to the true data distribution. As noted, the first energy-based model 170a may continue to “walk” the energy landscape of the noisy data distribution and draw samples therefrom until one or more criteria are met. For instance, the first energy-based model 170a may continue “walking” the energy landscape of the noisy data distribution until the sampling iteration k+1 if a threshold quantity of sampling iterations are performed at that point. Alternatively and/or additionally, the first energy-based model 170a may continue “walking” the energy landscape of the noisy data distribution until the sample yₖ₊₁ is drawn if the sample yₖ₊₁ exhibits a threshold energy value or a threshold likelihood of being in the noisy data distribution.

Training the first energy-based model 170a to approximate the data distribution of the protein sequences X may overfit the first energy-based model 170a to those specific sequences. This means that the first energy-based model 170a is able to accurately approximate the density of the regions in the data distribution that are within the immediate vicinity of these protein sequences X but not beyond. This phenomenon is illustrated in the top panel (A) of FIG. 4B, which shows that the gradient (or density estimation) of the data distribution being inaccurate for a large portion of the data distribution. That the first energy-based model 170a is unable to accurately approximate the density of large swaths of the data distribution may prevent the first energy-based model 170a from adequately exploring the data distribution during sampling, thus causing mode collapse in which the output of the first energy-based model 170a lacks the requisite diversity. Contrastingly, the bottom panel (B) of FIG. 4B shows that training the first energy-based model 170a based on noisy protein sequences Y may enable the first energy-based model 170a to accurately approximate the density of a larger portion of the data distribution. Accordingly, training the first energy-based model 170a to approximate a noisy data distribution may prevent overfitting as well as mode collapse.

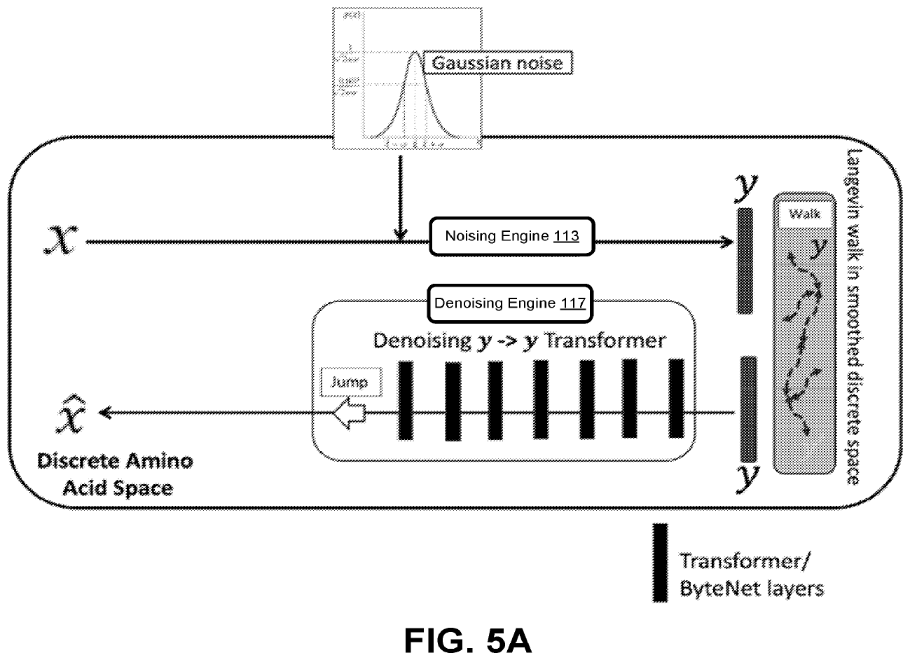

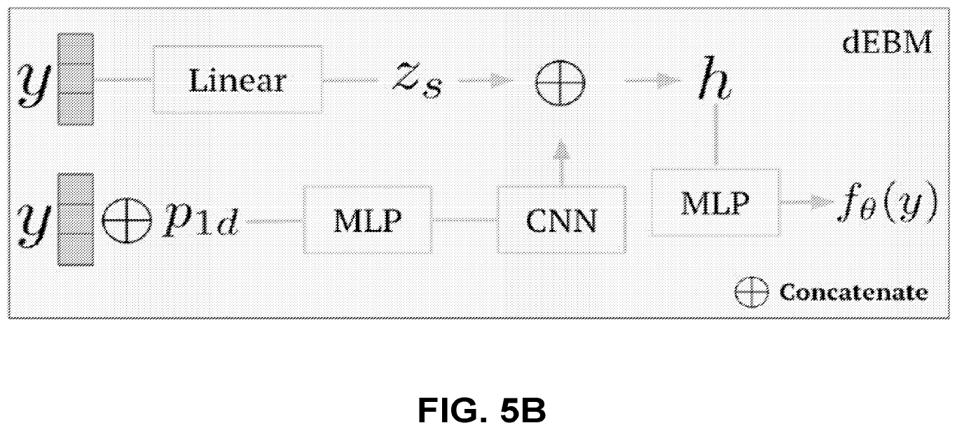

FIG. 5A depicts a schematic diagram illustrating an example of sampling from a smoothed discrete space, in accordance with some example embodiments. FIG. 5A shows one variation of the generative process in which the protein design computation model 115 operates in a smoothed discrete space, which is formed when the noising engine 113 adds noise (e.g., Gaussian noise and/or the like) to protein sequences. For example, FIG. 5A shows the protein sequences x and x as occupying a discrete space (e.g., discrete amino acid space) populated by individual (or discrete) protein sequences, each of which being represented by a constituent sequence of amino acid residues. The addition of noise (e.g., Gaussian noise and/or the like) to the protein sequence x may generate a first noisy sequence y. This may be tantamount to projecting the protein sequence x onto the aforementioned smoothed discrete space, which exhibits a smoother energy landscape than the initial discrete space. FIG. 5A shows that the first energy-based model 170a may sample from the smoothed discrete space by “walking” the smoothed discrete space from the noisy sequence y to a second noisy sequence y′. For instance, in some cases, the first energy-based model 170a may “walk” from the first noisy sequence y to the second noisy sequence y′ by modifying the first noisy sequence y over, in some cases, multiple successive iterations (e.g., gradient-based Markov Chain Monte Carlo (MCMC) sampling iterations and/or the like). The “walk” across the smoothed discrete space may be guided by the first energy function 175a (e.g., the gradient of the first energy function 175a). Accordingly, in some cases, the second noisy sequence y′ may include modifications that decrease the energy value of the second noisy sequence y′ relative to the first noisy sequence y, meaning that the second noisy sequence y′ is sampled from a higher density region of the noisy discrete space.

To further illustrate, FIG. 5B depicts a block diagram illustrating an example of a discrete energy-based model (dEBM) for implementing the first energy-based model 170a, in accordance with some example embodiments. As shown in FIG. 5B, the discrete energy-based model (dEBM) may ingest the first noisy sequence y, concatenate the first noisy sequence y with a positional encoding p (e.g., a one-dimensional positional encoding p_{1d}) before passing through a multilayer perceptron (MLP) and a convolutional neural network (CNN) to generate an output that is further concatenated with an embedding zₛ of the first noisy sequence y to form the hidden state h. This hidden state h is then passed through a multilayer perceptron (MLP) to return the energy function ƒ_{θ}(y).

Referring again to FIG. 5A, in some cases, the “walk” across the smoothed discrete space may include drawing multiple intermediate samples from the smoothed discrete space before reaching the second noisy sequence y′, with each intermediate sample being an incrementally lower energy configuration drawn from a higher density region of the smoothed discrete space. Moreover, the first energy-based model 170a may continue “walking” the smoothed discrete space until one or more criteria are met, at which point the second noisy sequence y′ may be denoised, for example, by the denoising engine 117, to generate the protein sequence z. As shown in FIG. 5A, the denoising of the second noisy sequence y′ may constitute a “jump” back to the discrete space. The protein sequence x is therefore a discrete protein sequence represented by a constituent sequence of amino acid residues.

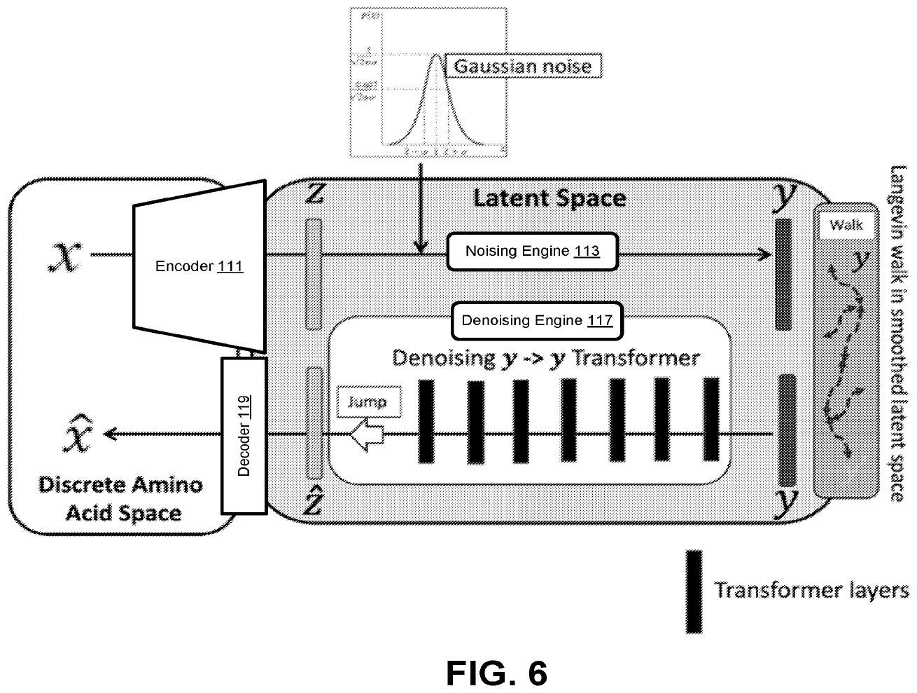

FIG. 6 depicts a schematic diagram illustrating an example of sampling from a smoothed latent space, in accordance with some example embodiments. FIG. 6 shows another variation of the generative process in which the protein design computation model 115 operates in a smoothed latent space, which is formed when the noising engine 113 adds noise (e.g., Gaussian noise and/or the like) to the embeddings of protein sequences generated by the encoder 111. In some cases, prior to adding noise (e.g., Gaussian noise and/or the like) to the protein sequence x, the encoder 111 may generate the embedding z of the protein sequence x by at least enriching the protein sequence x with additional information (e.g., structural information, environmental information, and/or the like). As shown in FIG. 6, the embedding z of the protein sequence x may occupy a latent space occupied by sequence embeddings instead of the discrete protein sequences found in the discrete space (e.g., discrete amino acid space). The noising engine 113 then generates the first noisy sequence y by adding noise (e.g., Gaussian noise and/or the like) to the embedding z of the protein sequence x. Adding noise to the embedding z of the protein sequence x instead of adding noise directly to the protein sequence x (as is the case in FIG. 5A) may further project the embedding z into a smoothed latent space populated by noisy sequence embeddings. The smoothed latent space may be more continuous and semantically meaningful that its discrete counterpart at least because the distance between two or more sequence embeddings in the smoothed latent space may reflect similarities (or dissimilarities) in protein sequence as well as conformation (or three-dimensional structure).

Referring again to FIG. 6, the first energy-based model 170a may “walk” the smoothed latent space while guided by the first energy function 175a. In the variation of the generative process shown in FIG. 6, the first energy-based model 170a may start the “walk” by modifying the first noisy sequence y which, as noted, is generated by the noising engine 113 adding noise to the embedding z of the protein sequence x. The first energy-based model 170a may “walk” the smoothed latent space by drawing one or more samples therefrom, with each sample including modifications that further decrease its energy value relative to one or more preceding samples. In some cases, the first energy-based models 170a may draw one or more intermediate samples between the first noisy sequence y and the second noisy sequence y′, with each intermediate sample being an incrementally lower energy configuration drawn from a higher density region of the smoothed latent space. Moreover, the first energy-based model 170a may continue “walking” the smoothed latent space until one or more criteria are met, at which point the denoising engine 117 may denoise the second noisy sequence y′ to generate the denoised embedding z before the protein sequence z is generated by the decoder 119 decoding the denoised embedding y′.

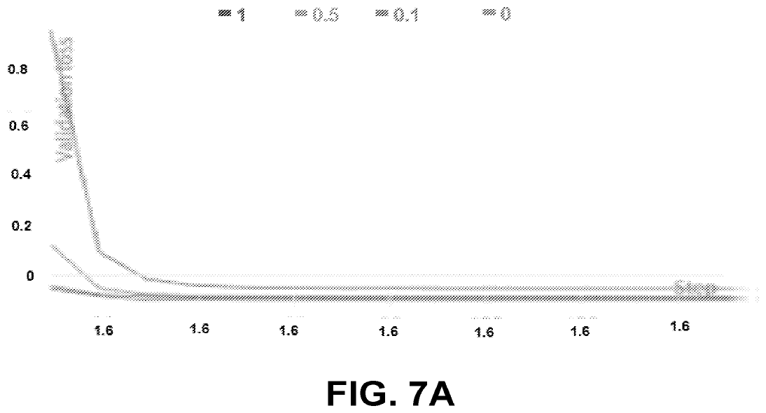

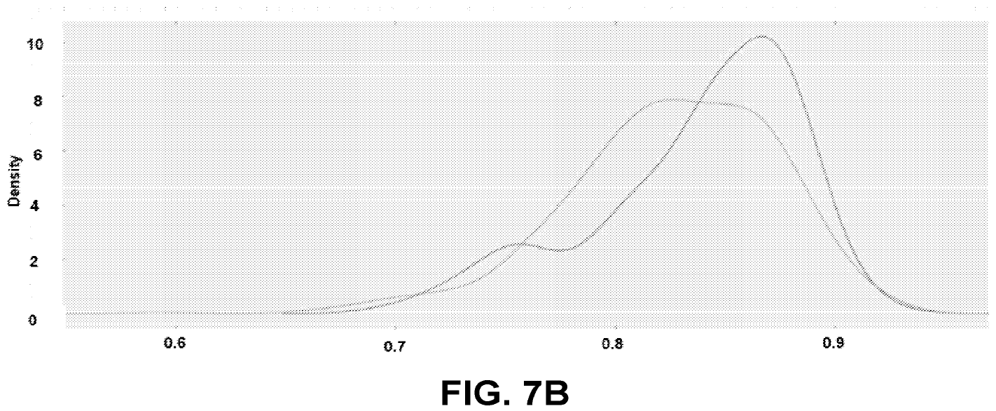

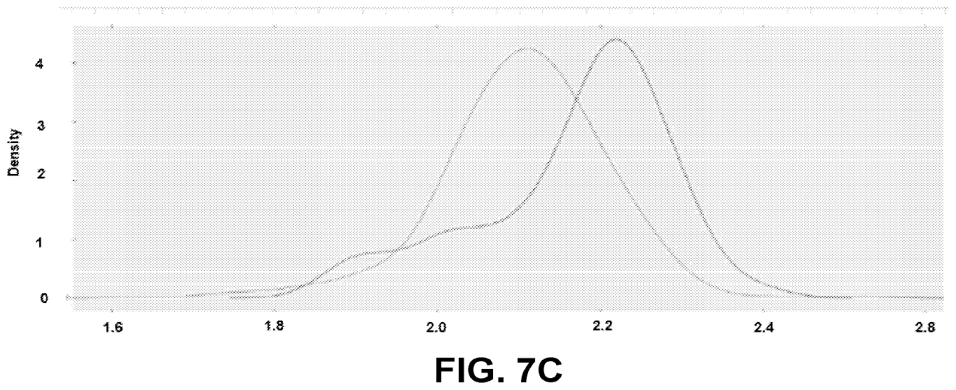

In some example embodiments, training the protein design computation model 115, particularly the first energy-based model 170a, based on a noisy training set containing noisy sample sequences prevents overfitting in the validation loss during maximum likelihood training. As shown in FIG. 7A, the loss of the first energy-based model 170a may converge quickly (e.g., at ˜50 training steps) and plateaus (e.g., for 100+ steps) without overfitting. Noising the sample sequences provides strong regularization that prevents overfitting. This effect is seen over a range of noise levels σ∈(0, 1.0). It should be appreciated that noise level σ=0 (no noise) is a special case that reflects the reconstruction accuracy of the encoder 111 and the decoder 119 or, alternatively, the baseline error that may be present in a sequence that undergoes encoding and decoding without the addition of any noise. In the absence of noise (e.g., σ=0), the true protein sequence and the protein sequence reconstructed by the decoder 119 from the embedding of the true protein sequence generated by the encoder 111 may exhibit very few edits (e.g., <3.5 on average) compared to clean sample sequences. These edits tend to occur in higher entropy positions (e.g., positions more likely to be occupied by different amino acid residues across different protein sequences) and may reflect the biophysical multiplicity observed in naturally occurring protein sequences (e.g., antibodies and/or the like). However, in the absence of noise (e.g., σ=0), sampling remains difficult as the energy landscape of the data distribution of the protein sequences lacks the smoothing afforded by the introduction of noise in the sample sequences.

In some example embodiments, the analysis engine 130 may determine, based at least on the output sequence 156, the performance of the protein design computation model 115 across a suite of “antibody likeness” (ab-likeness) metrics including, for example, labels derived from the amino acid sequence with Biopython, a sequence similarity score from sequence alignments with DIAMOND, Levenstein edit distances calculated with Edlib, a naturalness metric computed from the likelihoods of a masked language model pre-trained on antibody sequences, and/or the like. Sequence property metrics may be condensed into a single scalar metric by computing the normalized average Wasserstein distance, W_{property}, between the property distributions of the sample sequences in the training set and a validation set. The average total edit distance, E_{dist}, may summarize the novelty and diversity of samples compared to the validation set. The results summarized in Table 1 below show that with increasing variance σ, which controls the quantity of noise added to the sample sequences in the training set, better agreement is reached between the sample property distributions and the validation set. The average total edit distance also increases monotonically with increasing variance σ, reflecting an improvement in sequence novelty and diversity as well as mode exploration. Distributions of DIAMOND similarity metric (FIG. 7B) and naturalness metric (FIG. 7C) indicate that the protein design computation model 115 is able to generate natural sequences with reasonable similarity to the training sequences in the training set, while maintaining sequence diversity as well as sequence novelty.

TABLE 1

| | | |
|---|---|---|
| σ | W_{PROPERTY} ↓ | E_{dist} ↑ |

ψ = −∇E_{θ}(y)

| | | |
|---|---|---|
| 0 | 0.31 (0.31) | 2.3 (3.3) |
| 0.1 | 0.17 (0.20) | 4.9 (4.8) |
| 0.5 | 0.08 (0.10) | 16.5 (16.3) |
| 1.0 | 0.07 (0.08) | 33.5 (40.3) |

ψ = +∇E_{θ}(y)

| | | |
|---|---|---|
| 0 | 0.31 (0.31) | 2.3 (3.3) |
| 0.1 | 0.17 (0.18) | 5.0 (5.0) |
| 0.5 | 0.10 (0.11) | 16.5 (17.4) |
| 1.0 | 0.07 (0.10) | 30.9 (40.2) |

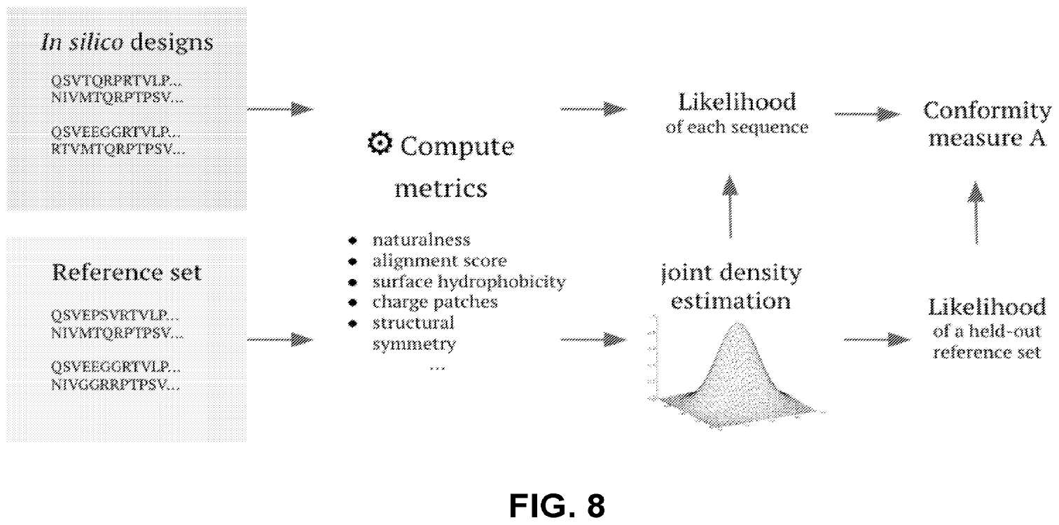

FIG. 8 depicts a schematic diagram illustrating a distributional conformity score based evaluation of the in silico protein designs generated by the protein design computation model 115 relative to a reference set of validation samples, in accordance with some example embodiments. In some example embodiments, the distributional conformity score may quantify the likelihood of an in silico protein design (e.g., the output sequence 162 in FIG. 1) with respect to a reference distribution, while maintaining novelty and diversity. In some cases, the distributional conformity score of the in silico protein design may correspond directly to the viability of the in silico protein design as a real, biophysically valid protein. In some cases, the probability of the in silico protein design conforming to a reference distribution may be evaluated using a conformal transducer system. For example, let [symbol] ∈ [symbol] ^{d}, [symbol] ∈ [symbol] , and Z= [symbol] × [symbol] , with x here denoting sample features and y denoting labels. The conformity measure A may be a measurable function that maps a sequence (z₁, . . . , zₙ)∈Zⁿ to a set of real numbers (α₁, . . . , αₙ) and is equivariant under permutations. Given a new sample z, the conformity measure A may quantify how similar z is to (z₁, . . . , zₙ). The conformal transducer can then be defined as a system of p-values where for each label y∈ [symbol] , a reference sequence (z₁, . . . , zₗ)∈Z^{l}, and a test sample x∈X, there is

$${p}^{y} := {p}^{y} ( {z}_{1} , … , {z}_{l} , ( x , y ) ) = \frac{1}{l\_ 1} {\sum }_{l - 1}^{l + 1} [ {α}_{{y}_{i}} < {α}_{{y}_{l + 1}} ]$$

wherein (α_{y}_{₁}, . . . , α_{y}_{ₗ}, α_{y}_{ₗ₊₁}):=A(z₁, . . . , zₗ, (x,y)). Intuitively, p^{y} is the fraction of in silico protein designs having a higher degree of conformity to the reference distribution than (x,y). In this context, the conformity measure A may be defined to be the likelihood under the joint density (e.g., computed using kernel density estimation) over various properties, such as biophysical properties and statistical properties (e.g., log-probability under a protein language model). Moreover, the reference distribution D may include a set of known protein sequences (e.g., antibodies) and the label y may represent a certain desirable property (e.g., expression, binding affinity, and/or the like).

As noted, in some cases, the performance of the protein design computation model 115 may be measured based on a suite of “antibody likeness” (ab-likeness) metrics. Sequence property metrics may be condensed into a single scalar metric by computing the distributional conformity score and the normalized average Wasserstein distance W_{property} between the property distribution of in silico protein designs and a validation set. The average total edit distance E_{dist} summarizes the novelty and diversity of the in silico protein designs, while internal diversity (IntDiv) is representative of the average total edit distance between the in silico protein designs as a group. As shown in Table 2 (below), the protein design computation model 115 achieved strong antibody likeness (ab-likeness) when the noise level is increased, for example, to σ≥0.5. Moreover, both implementations of the protein design computation model 115 (e.g., energy-based sampling and score-based sampling) achieved faster sampling time and lower memory footprint than conventional methodologies such as latent sequence diffusion (SeqVDM), score-based model with energy parameterization (DEEN), and a pre-trained large language model (GPT 3.5).

TABLE 2

| Model | W_{property} ↓ | Unique ↑ | E_{dist} ↑ | IntDiv ↑ | DCS ↑ |
|---|---|---|---|---|---|
| dWJS | 0.056 | 1.0 | 58.4 | 55.3 | 0.38 |
| (energy-based) | | | | | |
| dWJS | 0.065 | 0.97 | 62.7 | 65.1 | 0.49 |
| (score-based) | | | | | |
| SeqVDM | 0.062 | 1.0 | 60.0 | 57.4 | 0.40 |
| DEEN | 0.087 | 0.99 | 50.9 | 42.7 | 0.41 |
| GPT 3.5 | 0.14 | 0.66 | 55.4 | 46.1 | 0.23 |

The performance of the protein design computation model 115 in generating natural, novel, and diverse protein designs was also evaluated in vitro, with the protein design computation model 115 achieving a 97.47% in vitro success rate, with 270 of 277 in silico antibody designs being successfully expressed and purified in the laboratory. These results are shown in Table 3 below.

TABLE 3

| Model | pₑₓₚᵣₑₛₛᵢₒₙ ↑ |
|---|---|
| dWJS (score-based) | 1.0 |
| dWJS (energy-based) | 0.97 |
| EBM | 0.42 |

Furthermore, the performance of the protein design computation model 115 in generating functional protein designs was evaluated in vitro, with the protein design computation model 115 generating a greater percentage of binding antibodies than other methodologies such as such as latent sequence diffusion (SeqVDM), a pre-trained large language model (GPT 4), a transformer model, and an equivariant graph neural network (EGNN). These results are shown in Table 4 below.

TABLE 4

| Model | p_{bind} ↑ | total_{bind} ↑ | improved_{bind} ↑ |
|---|---|---|---|
| dWJS (energy-based) | 0.96 | 0.34 | 0.35 |
| dWJS (score-based) | 0.95 | N/A | N/A |
| SeqVDM | 0.75 | 0.19 | 0.0 |
| GPT4 | 0.74 | N/A | N/A |
| Transformer | 0.60 | N/A | N/A |
| EGNN | 0.58 | N/A | N/A |

The performance of the protein design computation model 115 operating in the latent space (lWJS) at different noise levels (sigma) instead of the discrete space (dWJS) is also evaluated based on the metrics Wasserstein distance (W_{property}), uniqueness, edit distance (E_{dist}), and internal diversity (IntDiv). Table 5 below summarizes the results for 2000 in silico antibody heavy chain designs generated based on 20 de novo seed sequences.

TABLE 5

| Model | W_{property} | Unique | E_{dist} | IntDiv |
|---|---|---|---|---|
| dWJS (energy-based) | 0.056 | 1.0 | 58.4 | 55.3 |
| dWJS (score-based) | 0.065 | 0.97 | 62.7 | 65.1 |
| lWJS (score-based) sigma = 2.5 | 0.053 | 1.0 | 56.6 | 54.1 |
| lWJS (score-based) sigma = 5.0 | 0.054 | 1.0 | 51.9 | 46.1 |
| lWJS (score-based) sigma = 7.0 | 0.052 | 1.0 | 54.2 | 49.5 |
| lWJS (score-based) sigma = 10.0 | 0.055 | 1.0 | 52.1 | 47.2 |
| lWJSpAb (score-based) sigma = | 0.051 | 1.0 | 48.6 | 35.2 |
| 2.5 | | | | |

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A computer-implemented method, comprising: generating a first training set to include a plurality of noisy sample sequences, each noisy sample sequence in the first training set being generated by at least adding noise to a corresponding sample sequence from a first data distribution; training a protein design computation model by at least applying the protein design computation model to generate one or more output sequences, and adjusting the protein design computation model to reduce a difference between the one or more generated output sequences and the plurality of noisy sample sequences in the first training set; applying the trained protein design computation model to generate an output sequence by at least modifying an input sequence.

Item 2: The method of Item 1, wherein the protein design computation model includes a first energy-based model (EBM).

Item 3: The method of Item 2, wherein the training of the protein design computation model includes adjusting a plurality of parameters of the first energy-based model parameterizing an energy function of the first energy-based model.

Item 4: The method of Item 3, wherein the plurality of parameters are adjusted such that an energy value determined by the energy function corresponds to a likelihood of the one or more generated output sequences within the first data distribution.

Item 5: The method of any of Items 3 to 4, wherein the plurality of parameters are adjusted such that the energy function outputs a lower energy value for a first generated output sequence that is more similar to the plurality of noisy samples in the first training set than a second generated output sequence that is less similar to the plurality of noisy samples in the first training set.

Item 6: The method of any of Items 3 to 5, wherein the training of the protein design computation model includes applying the first energy-based model having a first adjustment to generate a first modified sequence, applying the first energy-based model having a second adjustment to generate a second modified sequence, and upon determining that the first modified sequence is more similar to the plurality of noisy samples in the first training set than the second modified sequence, further modifying the first energy-based model having the first adjustment instead of the second adjustment.

Item 7: The method of Item 6, wherein the first energy-based model is further adjusted until one or more criteria are met, and wherein the one or more criteria include at least one of (i) having performed a threshold quantity of iterations of adjustments to the first energy-based model and (ii) the second modified sequence exhibiting a threshold similarity to the plurality of noisy samples in the first training set.

Item 8: The method of any of Items 2 to 7, wherein the protein design computation model further includes a second energy-based model (EBM).

Item 9: The method of Item 8, further comprising: generating a second training set including a plurality of sample sequences from a second data distribution; determining a first adjustment to the first energy-based model that reduces a first difference between a first output sequence generated by the first energy-based model and the plurality of noisy sample sequences in the first training set; determining a second adjustment to the second energy-based model that reduces a second difference between a second output sequence generated by the second energy-based model and the plurality of sample sequences in the second data distribution; and training the first energy-based model by at least applying, to the first energy-based model, a third adjustment determined based on the first adjustment and the second adjustment.

Item 10: The method of Item 9, wherein the third adjustment corresponds to a sum or a weighted sum of the first adjustment and the second adjustment.

Item 11: The method of any of Items 1 to 10, further comprising: encoding each sample sequence from the first data distribution to generate an embedding of each sample sequence; and generating the plurality of noisy sample sequences in the first training set by at least adding noise to the embedding of each sample sequence.

Item 12: The method of Item 11, wherein each sample sequence from the first data distribution is encoded by being enriched with additional information.

Item 13: The method of Item 12, wherein the additional information includes structural information that identifies, for each constituent amino acid residue, one or more neighboring amino acid residue in three-dimensional space.

Item 14: The method of any of Items 1 to 13, wherein the trained protein design computation model generates the output sequence by at least generating a noisy input sequence by at least adding noise to the input sequence, applying an energy-based model to generate a noisy output sequence by at least modifying, based at least on an energy function of the energy-based model, the noisy input sequence, and generating the output sequence by at least denoising the modified noisy output sequence generated by the energy-based model.

Item 15: The method of any of claims 1 to 14, wherein the trained protein design computation model generates the output sequence by at least generating an embedding of the input sequence by at least encoding the input sequence, generating a noisy embedding of the input sequence by at least adding noise to the embedding of the input sequence, applying an energy-based model to generate a modified noisy embedding by at least modifying, based at least on an energy function of the energy-based model, the noisy embedding of the input sequence, denoising the noisy embedding to generate a denoised embedding, and generating the output sequence by at least denoising the noisy embedding.

Item 16: The method of Item 15, wherein the embedding of the input sequence is generated by at least generating, for each amino acid residue in the input sequence, a token encoding an identity of the amino acid residue.

Item 17: The method of any of claims 15 to 16, wherein the embedding of the input sequence is generated by at least generating one or more structural tokens identifying, for at least one amino acid residue in the input sequence, one or more neighboring amino acid residue in three-dimensional space.

Item 18: The method of any of Items 1 to 17, wherein the trained protein design computation model modifies the input sequence by at least one of (i) inserting an amino acid residue, (ii) deleting an amino acid residue, and (iii) changing an identity of an amino acid residue in the input sequence.

Item 19: The method of any of Items 1 to 18, further comprising: generating a fixed-length representation of the input sequence; and applying the trained protein design computation model to generate the output sequence by at least modifying the fixed length representation of the input sequence.

Item 20: The method of Item 19, wherein the fixed-length representation of the input sequence is generated by at least aligning each amino acid residue in the input sequence to a fixed set of structural roles such that each amino acid residue in the input sequence is assigned an integer position corresponding to a structural role of the amino acid residue, and inserting a gap character at one or more positions where the input sequence fails to include an amino acid residue having a corresponding structural role.

Item 21: The method of any of Items 1 to 20, wherein the difference between the one or more generated output sequences and the plurality of noisy sample sequences is quantified by one or more of an antibody likeness metric, an edit distance, and a naturalness metric.

Item 22: A computer-implemented method, comprising: identifying an input sequence having a plurality of amino acid residues; generating a noisy embedding of the input sequence by at least adding noise to the input sequence; modifying the noisy embedding of the input sequence by at least applying a protein design computation model trained to approximate a data distribution of protein sequences exhibiting one or more desirable properties, the protein design computation model modifying the noisy embedding of the input sequence to increase a likelihood of a modified noisy embedding resulting therefrom being in the data distribution; and generating an output sequence by at least denoising the modified noisy embedding generated by the protein design computation model.

Item 23: The method of Item 22, further comprising: encoding the input sequence to generate an embedding of the input sequence; generating the noisy embedding of the input sequence by at least adding noise to the embedding of the input sequence; and generating the output sequence by decoding a denoised embedding generated by the denoising of the modified noisy embedding.

Item 24: The method of Item 23, wherein the input sequence is encoded by at generating, for each amino acid residue in the input sequence, a token encoding an identity of the amino acid residue.

Item 25: The method of any of Items 23 to 24, wherein the input sequence is encoded by at least generating one or more tokens encoding a relative position of each amino acid residue within the input sequence.

Item 26: The method of any of Items 23 to 24, wherein the input sequence is encoded by at least generating one or more structural tokens identifying, for at least one amino acid residue in the input sequence, one or more neighboring amino acid residue in three-dimensional space.

Item 27: The method of any of claims 22 to 26, wherein the modifying of the noisy embedding includes applying an energy-based model (EBM) trained to approximate the data distribution to modify the noisy embedding of the input sequence and generate a first modified noisy embedding, applying the energy-based model (EBM) to modify the noisy embedding of the input sequence and generate a second modified noisy embedding, applying an energy function parameterized by the energy-based model (EBM) to determine a first energy value of the first modified noisy embedding and a second energy value of the second modified noisy embedding, and applying the energy-based model (EBM) to further modify, based at least on the first energy value and the second energy value, the first modified noisy embedding instead of the second modified noisy embedding.

Item 28: The method of Item 27, wherein the energy-based model (EBM) is applied to further modify the first modified noisy embedding until one or more criteria are met.

Item 29: The method of Item 28, wherein the one or more criteria include at least one of (i) having performed a threshold quantity of iterations of modifications to the noisy embedding of the input sequence and (ii) the first energy value of the first modified noisy embedding satisfying one or more thresholds.

Item 30: The method of any of Items 27 to 28, wherein the energy-based model (EBM) is applied to further modify the first modified nosy embedding instead of the second modified noisy embedding based at least on the first energy value and the second energy value indicating that the first modified noisy embedding has a higher likelihood of being in the data distribution than the second modified noisy embedding.

Item 31: The method of any of Items 27 to 30, wherein the energy-based model (EBM) is applied to further modify the first modified nosy embedding instead of the second modified noisy embedding based at least on the first energy value and the second energy value indicating that the first modified noisy embedding is sampled from a higher density region of the data distribution than the second modified noisy embedding.

Item 32: The method of any of Items 22 to 31, further comprising: generating a fixed-length representation of the input sequence; and generating, based at least on the fixed-length representation of the input sequence, the noisy embedding of the input sequence.

Item 33: The method of Item 32, wherein the fixed-length representation of the input sequence is generated by at least aligning each amino acid residue in the input sequence to a fixed set of structural roles such that each amino acid residue in the input sequence is assigned an integer position corresponding to a structural role of the amino acid residue, and inserting a gap character at one or more positions where the input sequence fails to include an amino acid residue having a corresponding structural role.

Item 34: The method of any of Items 32 to 33, wherein the protein design computation model modifies the noisy embedding of the input sequence by at least one changing an identity of one or more amino acid residues in the input sequence, deleting an amino acid residue occupying a position within the fixed-length representation of the input sequence by at least replacing the amino acid residue with a gap character, and inserting an amino acid residue at a position within the fixed-length representation of the input sequence by at least replacing a gap residue occupying the position with the amino acid residue.

Item 35: The method of any of Items 22 to 34, wherein the one or more desirable properties include at least one of expression, affinity, specificity, stability, non-immunogenicity, human-ness, absence of self-association, and lack of chemical liabilities.

Item 36: The method of any of Items 22 to 35, wherein the input sequence is a known protein sequence or a noise sequence comprising a random sequence of amino acid residues.

Item 37: A system, comprising: at least one data processor; and at least one memory storing instructions, which when executed by the at least one data processor, result in operations comprising the method of any of Items 1 to 21 or the method of any of Items 22 to 36.

Item 38: A non-transitory computer readable medium storing instructions, which when executed by at least one data processor, result in operations comprising the method of any of Items 1 to 21 or the method of any of Items 22 to 36.

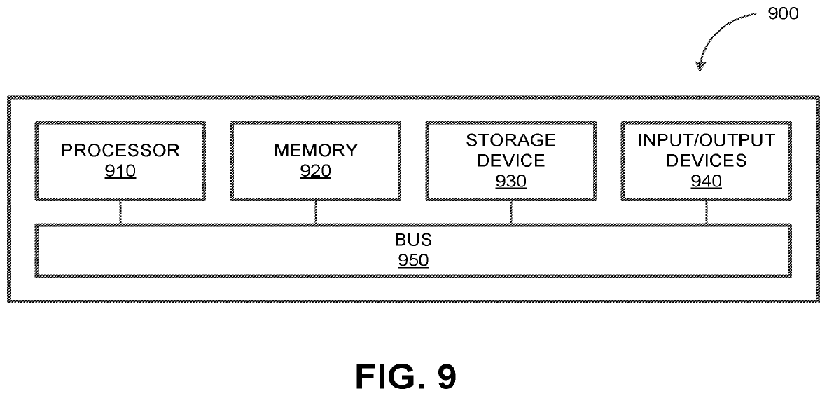

FIG. 9 depicts a block diagram illustrating an example of a computing system 900, in accordance with some example embodiments. Referring to FIGS. 1-9, the computing system 900 may be used to implement the protein design engine 110, the analysis engine 120, the client device 130, and/or any components therein.

As shown in FIG. 9, the computing system 900 can include a processor 910, a memory 920, a storage device 930, and input/output devices 940. The processor 910, the memory 920, the storage device 930, and the input/output devices 940 can be interconnected via a system bus 950. The processor 910 is capable of processing instructions for execution within the computing system 900. Such executed instructions can implement one or more components of, for example, the protein design engine 110, the analysis engine 120, the client device 130, and/or the like. In some example embodiments, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a hard disk device, an optical disk device, or a tape device, or other suitable persistent storage means. The input/output device 940 provides input/output operations for the computing system 900. In some example embodiments, the input/output device 940 includes a keyboard and/or pointing device. In various implementations, the input/output device 940 includes a display unit for displaying graphical user interfaces.

According to some example embodiments, the input/output device 940 can provide input/output operations for a network device. For example, the input/output device 940 can include Ethernet ports or other networking ports to communicate with one or more wired and/or wireless networks (e.g., a local area network (LAN), a wide area network (WAN), the Internet).

In some example embodiments, the computing system 900 can be used to execute various interactive computer software applications that can be used for organization, analysis and/or storage of data in various formats. Alternatively, the computing system 900 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor, and
at least one memory storing instructions, which when executed by the at least one data processor, result in operations comprising:
receiving a data distribution of protein sequences, each of the protein sequences exhibiting one or more desired properties;
identifying a sample sequence from the data distribution of protein sequences;
generating a noisy sample sequence by at least adding noise to the sample sequence;
generating a training dataset including a plurality of noisy sample sequences, where the plurality of noisy sample sequences form a noisy data distribution, and where the training dataset is generated to include the noisy sample sequence;
training a protein design computation model to approximate the noisy data distribution by at least
applying the protein design computation model to generate a sample output sequence, and
adjusting the protein design computation model to reduce a difference between the sample output sequence generated by the protein design computation model and the plurality of noisy sample sequences in the training dataset; and
receiving an input sequence;
applying the trained protein design computation model to modify the input sequence, where modifying the input sequence includes generating an output sequence exhibiting the one or more properties.

2. The system of claim 1, wherein the protein design computation model includes an energy-based model (EBM).

3. The system of claim 2, wherein the training of the protein design computation model includes adjusting a plurality of parameters of the energy-based model, and wherein the plurality of parameters parameterize an energy function 4 associated with the energy-based model.

4. The system of claim 3, wherein the plurality of parameters are adjusted such that an energy value determined by the energy function corresponds to a likelihood of the one or more sample output sequences within the data distribution of the training dataset.

5. The system of claim 3, wherein the plurality of parameters are adjusted such that the energy function outputs a lower energy value for a sample output sequence that is more similar to the plurality of noisy samples in the training dataset than another sample output sequence that is less similar to the plurality of noisy samples in the training dataset.

6. The system of claim 3, wherein the training of the protein design computation model includes
applying the energy-based model having a first adjustment to generate a first modified sequence,
applying the energy-based model having a second adjustment to generate a second modified sequence, and
upon determining that the first modified sequence is more similar to the plurality of noisy samples in the training dataset than the second modified sequence, further modifying the energy-based model having the first adjustment instead of the energy-based model having the second adjustment.

7. The system of claim 6, wherein the energy-based model is further adjusted until one or more criteria are met, and wherein the one or more criteria include at least one of (i) having performed a threshold quantity of iterations of adjustments to the energy-based model and (ii) the first modified sequence and/or the second modified sequence exhibiting a threshold similarity to the plurality of noisy samples in the training dataset.

8. The system of claim 2, wherein the protein design computation model further includes an additional energy-based model (EBM).

9. The system of claim 8, further comprising:
generating an additional training dataset including a plurality of sample sequences from a different data distribution;
determining a first adjustment to the energy-based model that reduces a difference between a first output sample sequence generated by the energy-based model and the plurality of noisy sample sequences in the training dataset;
determining a second adjustment to the additional energy-based model that reduces a difference between a second output sample sequence generated by the additional energy-based model and the plurality of sample sequences in the additional training dataset; and
training the energy-based model by at least applying, to the energy-based model, a third adjustment determined based on the first adjustment and the second adjustment.

10. The system of claim 9, wherein the third adjustment corresponds to a sum or a weighted sum of the first adjustment and the second adjustment.

11. The system of claim 1, further comprising:
encoding each sample sequence from the data distribution to generate an embedding of each sample sequence, wherein the encoding includes enriching with structural information that identifies, for at least one amino acid residue in each sample sequence, one or more neighboring amino acid residue in three-dimensional space; and
generating the plurality of noisy sample sequences in the training dataset by at least adding noise to the embedding of each sample sequence.

12. (canceled)

13. (canceled)

14. The system of claim 1, wherein the trained protein design computation model generates the output sequence by at least
generating a noisy input sequence by at least adding noise to the input sequence,
applying an energy-based model to generate a noisy output sequence by at least modifying, based at least on an energy function of the energy-based model, the noisy input sequence, and
generating the output sequence by at least denoising the modified noisy output sequence generated by the energy-based model.

15. The system of claim 1, wherein the trained protein design computation model generates the output sequence by at least
generating an embedding of the input sequence by at least encoding the input sequence,
generating a noisy embedding of the input sequence by at least adding noise to the embedding of the input sequence,
applying an energy-based model to generate a modified noisy embedding by at least modifying, based at least on an energy function of the energy-based model, the noisy embedding of the input sequence,
denoising the noisy embedding to generate a denoised embedding, and
generating the output sequence by at least denoising the noisy embedding.

16. (canceled)

17. The system of claim 15, wherein the embedding of the input sequence is generated by at least
generating one or more structural tokens identifying, for at least one amino acid residue in the input sequence, one or more neighboring amino acid residue in three-dimensional space.

18. (canceled)

19. The system of claim 1, further comprising:
generating a fixed-length representation of the input sequence by at least
aligning each amino acid residue in the input sequence to a fixed set of structural roles such that each amino acid residue in the input sequence is assigned an integer position corresponding to a structural role of the amino acid residue, and
inserting a gap character at one or more positions where the input sequence fails to include an amino acid residue having a corresponding structural role; and
applying the trained protein design computation model to generate the output sequence by at least modifying the fixed length representation of the input sequence.

20. (canceled)

21. The system of claim 1, wherein the difference between the one or more generated output sequences and the plurality of noisy sample sequences is quantified by one or more of an antibody likeness metric, an edit distance, and a naturalness metric.

22. A system, comprising:
at least one data processor; and
at least one memory storing instructions, which when executed by the at least one data processor, result in operations comprising:
identifying an input sequence having a plurality of amino acid residues;
generating a noisy embedding of the input sequence by at least adding noise to the input sequence;
modifying the noisy embedding of the input sequence by at least applying a protein design computation model trained to approximate a data distribution of protein sequences exhibiting one or more properties, the protein design computation model modifying the noisy embedding of the input sequence to increase a likelihood of a modified noisy embedding resulting from the modifying being in the data distribution; and
generating an output sequence by at least denoising the modified noisy embedding generated by the protein design computation model.

23. The system of claim 22, further comprising:
encoding the input sequence to generate an embedding of the input sequence;
generating the noisy embedding of the input sequence by at least adding noise to the embedding of the input sequence; and
generating the output sequence by decoding a denoised embedding generated by the denoising of the modified noisy embedding.

24. The system of claim 23, wherein the input sequence is encoded by at generating, for each amino acid residue in the input sequence, a token encoding an identity of the amino acid residue.

25. The system of claim 23, wherein the input sequence is encoded by at least generating one or more tokens encoding a relative position of each amino acid residue within the input sequence.

26. The system of claim 23, wherein the input sequence is encoded by at least generating one or more structural tokens identifying, for at least one amino acid residue in the input sequence, one or more neighboring amino acid residue in three-dimensional space.

27. The system of claim 22, wherein the modifying of the noisy embedding includes
generating a first modified noisy embedding by applying an energy-based model (EBM) trained to approximate the data distribution to modify the noisy embedding of the input sequence,
generating a second modified noisy embedding by applying the energy-based model (EBM) to modify the noisy embedding of the input sequence,
applying an energy function parameterized by a plurality of parameters of the energy-based model (EBM) to determine an energy value of the first modified noisy embedding and an energy value of the second modified noisy embedding, and
applying the energy-based model (EBM) to further modify, based at least on the energy value of the first modified noisy embedding and the energy value of the second modified noisy embedding, at least one of the first modified noisy embedding and the second modified noisy embedding.

28. The system of claim 27, wherein the energy-based model (EBM) is applied to further modify the at least one of the first modified noisy embedding and the second modified noisy embedding until one or more criteria are met, and wherein the one or more criteria include at least one of (i) having performed a threshold quantity of iterations of modifications to the noisy embedding of the input sequence and (ii) the energy value of the first modified noisy embedding and/or the energy value of the second modified noisy embedding satisfying one or more thresholds.

29. (canceled)

30. The system of claim 27, wherein the energy-based model (EBM) is applied to further modify the first modified nosy embedding instead of the second modified noisy embedding based on the energy value of the first modified noisy embedding and the energy value of the second modified noisy embedding indicating at least one of (i) the first modified noisy embedding having a higher likelihood of being in the data distribution than the second modified noisy embedding, and (ii) the first modified noisy embedding being sampled from a higher density region of the data distribution than the second modified noisy embedding.

31. (canceled)

32. The system of claim 22, further comprising:
generating a fixed-length representation of the input sequence by at least
aligning each amino acid residue in the input sequence to a fixed set of structural roles such that each amino acid residue in the input sequence is assigned an integer position corresponding to a structural role of the amino acid residue, and
inserting a gap character at one or more positions where the input sequence fails to include an amino acid residue having a corresponding structural role; and
generating, based at least on the fixed-length representation of the input sequence, the noisy embedding of the input sequence.

33. (canceled)

34. The system of claim 32, wherein the protein design computation model modifies the noisy embedding of the input sequence by at least one
changing an identity of one or more amino acid residues in the input sequence,
deleting an amino acid residue occupying a position within the fixed-length representation of the input sequence by at least replacing the amino acid residue with a gap character, and
inserting an amino acid residue at a position within the fixed-length representation of the input sequence by at least replacing a gap residue occupying the position with the amino acid residue.

35. The system of claim 22, wherein the one or more properties include at least one of expression, affinity, specificity, stability, non-immunogenicity, human-ness, absence of self-association, and lack of chemical liabilities.

36. The system of claim 22, wherein the input sequence is a known protein sequence or a noise sequence comprising a random sequence of amino acid residues.

37. (canceled)

38. (canceled)

39. A computer-implemented method, comprising:
receiving a data distribution of protein sequences, each of the protein sequences exhibiting one or more desired properties;
identifying a sample sequence from the data distribution of protein sequences;
generating a noisy sample sequence by at least adding noise to the sample sequence;
generating a training dataset including a plurality of noisy sample sequences, where the plurality of noisy sample sequences form a noisy data distribution, and where the training dataset is generated to include the noisy sample sequence;
training a protein design computation model to approximate the noisy data distribution by at least
applying the protein design computation model to generate a sample output sequence, and
adjusting the protein design computation model to reduce a difference between the sample output sequence generated by the protein design computation model and the plurality of noisy sample sequences in the training dataset; and
receiving an input sequence;
applying the trained protein design computation model to modify the input sequence, where modifying the input sequence includes generating an output sequence exhibiting the one or more properties.

40. A computer-implemented method, comprising:
receiving a data distribution of protein sequences, each of the protein sequences exhibiting one or more desired properties;
identifying a sample sequence from the data distribution of protein sequences;
generating a noisy sample sequence by at least adding noise to the sample sequence;
generating a training dataset including a plurality of noisy sample sequences, where the plurality of noisy sample sequences form a noisy data distribution, and where the training dataset is generated to include the noisy sample sequence;
training a protein design computation model to approximate the noisy data distribution by at least
applying the protein design computation model to generate a sample output sequence, and
adjusting the protein design computation model to reduce a difference between the sample output sequence generated by the protein design computation model and the plurality of noisy sample sequences in the training dataset; and
receiving an input sequence;
applying the trained protein design computation model to modify the input sequence, where modifying the input sequence includes generating an output sequence exhibiting the one or more properties.
